(19) United States
(12) Patent Application Publication
(10) Pub. No.: US 2025/0369011 A1
(43) Pub. Date: Dec. 4, 2025

(54) INSULIN PROMOTER FOR GENE THERAPY FOR TYPE 2 DIABETES MELLITUS

(71) Applicant: University of Pittsburgh - Of the Commonwealth System of Higher Education, Pittsburgh, PA (US)

(72) Inventor: George Gittes, Gibsonia, PA (US)

(73) Assignee: University of Pittsburgh - Of the Commonwealth System of Higher Education, Pittsburgh, PA (US)

(21) Appl. No.: 18/867,423

(22) PCT Filed: May 18, 2023
(86) PCT No.: PCT/US2023/067183

§ 371 Date: Nov. 19, 2024

Related U.S. Application Data
(60) Provisional application No. 63/344,226, filed on May 20, 2022.

(51) Int. Cl. C12N 15/86; A61K 38/00; A61K 48/00; C07K 14/47

(52) U.S. Cl. CPC C12N 15/86; A61K 48/0058; C07K 14/4705; A61K 38/00; C12N 2750/14143

(57) ABSTRACT

Methods are disclosed for treating a subject with type 2 diabetes. The methods include administering to the subject a therapeutically effective amount of a vector including an insulin promoter operably linked to a nucleic acid molecule encoding heterologous Pancreas duodenal homeobox protein (Pdx) 1 and MafA. In some embodiments, the vector does not encode Neurogenin 3 (Ngn3) and wherein the subject is not administered any other nucleic acid encoding Ngn3. The vector can be administered intraductally into a pancreatic duct of the subject. Compositions are disclosed that include a) a viral vector comprising an insulin promoter operably linked to a nucleic acid molecule encoding Pdx1 and a nucleic acid encoding MafA, wherein the vector does not encode Ngn3; b) a buffer; and c) a contrast dye for endoscopic retrograde cholangiopancreatography. These compositions are of use in any of the methods disclosed herein, and can be used to the improve hyperglucagonemia, insulin sensitivity, and/or glucose homeostasis in the subject.

### CROSS REFERENCE TO RELATED APPLICATIONS

This claims the benefit of U.S. Provisional Application No. 63/344,226, filed May 20, 2022, which is incorporated herein by reference.

### FIELD OF THE DISCLOSURE

This relates to the field of diabetes, specifically to the intraductal administration of a viral vector comprising an insulin promoter operably linked to a nucleic acid molecule encoding pancreas duodenal homeobox protein (Pdx) 1 and MafA to treat type 2 diabetes mellitus (T2DM).

### REFERENCE TO AN ELECTRONIC SEQUENCE LISTING

The contents of the electronic sequence listing (Sequences.xml, which has a size of: 13000 bytes and a Date of Creation of May 18, 2023) is herein incorporated by reference in its entirety.

### BACKGROUND

Diabetes is a significant health problem in the United States and worldwide. According to the CDC National Diabetes Statistics Report (2017), in 2015 the prevalence of diabetes in the USA was 30.3 million (9.4%), and 84.1 million Americans aged 18 and older had prediabetes. Diabetes remains the 7th leading cause of death in the USA (Mayer-Davis et al., N Engl J Med. 2017; 376(15):1419-29). With the worsening obesity epidemic, the incidence of T2DM has been rising (Mayer-Davis et al., N Engl J Med. 2017; 376(15):1419-29). Obesity is an insulin-resistant state that places significant stress on the pancreatic B-cells as they augment insulin secretion to overcome the insulin resistance. As long as the β-cells can increase their insulin secretion sufficiently to overcome the insulin resistance, glucose tolerance remains normal (Gastaldeli et al., Diabetologia. 2004; 47(1):31-9). With long-standing insulin resistance, there can be early B-cell dysfunction (Abdul-Ghani et al., Am J Physiol Endocrinol Metab. 2008; 295(2):E401-6) and progressive β-cell loss leading to the onset of overt diabetes (Hanley et al., Endocrinology. 2010; 151(4):1462-72). Disclosed herein are methods for treating T2DM.

### SUMMARY OF THE DISCLOSURE

It is disclosed herein that a viral vector, such as an adenoviral vector or an adeno-associated viral vector comprising an insulin promoter operably linked to a nucleic acid molecule encoding pancreas duodenal homeobox protein (Pdx1) and musculoaponeurotic fibrosarcoma oncogene homolog A (MafA) can be used to treat T2DM in a subject. The disclosed methods can be used to improve hyperglucagonemia, improve insulin sensitivity, and/or improve glucose homeostasis.

In some embodiments, the viral vector can be delivered to the subject using endoscopic retrograde cholangiopancreatography (ERCP).

In further aspects, the subject is not administered Neurogenin 3 (Ngn3) or a nucleic acid encoding Ngn3.

In more aspects a composition is disclosed that includes a) an adeno-associated virus vector comprising an insulin promoter operably linked to a nucleic acid molecule encoding Pdx1 and a nucleic acid molecule encoding MafA; b) a buffer; and c) a contrast dye for endoscopic retrograde cholangiopancreatography. In specific non-limiting examples, the vector does not encode Ngn3. These compositions are of use in any of the methods disclosed herein, such as for treating a subject with T2DM. The compositions can be used to improve hyperglucagonemia, improve insulin sensitivity, and/or improve glucose homeostasis in a subject, such as a subject with T2DM. In specific non-limiting examples, these compositions are formulated for intraductal administration.

The foregoing and other features and advantages of the invention will become more apparent from the following detailed description of several aspects which proceeds with reference to the accompanying figures.

### BRIEF DESCRIPTION OF THE FIGURES

FIGS. 1A-1F. A: Body weights of mice. B, C: Intraperitoneal glucose tolerance test (IPGTT) at 2 weeks (B), area under curve (AUC) analysis (C). D: Glucose stimulated insulin secretion (GSIS) during the IPGTT. E: Glucagon measurements during the IPGTT. F: homeostatic model assessment for insulin resistance (HOMA-IR). Data are presented as mean±S.D. Statistical analysis was done by repeated measure ANOVA for GSIS. One-way ANOVA was used for A, C, E and F. Holm Sidak's test was used for multiple comparisons. * p<0.05, ** p<0.01, *** p<0.001, **** p<0.0001, and ns=no significance.

FIGS. 2A-2H. A: Body weights of mice. B, C: IPGTT at 2 weeks (B), AUC analysis (C). D: GSIS during the IPGTT. E: Glucagon measurements during the IPGTT. F: HOMA-IR. G: ex-vivo GSIS for isolated islets (30 islets/mouse). H: comparison of Ex-vivo GSIS between AAV-CMV-PM and AAV-RIP-PM. Data are presented as mean±S.D. Statistical analysis was done by repeated measure ANOVA for GSIS. One-way ANOVA was used for A, C, E and F. Holm Sidak's test was used for multiple comparisons. * p<0.05, ** p<0.01, *** p<0.001, **** p<0.0001, and ns=no significance.

FIG. 3. Map of construct used in the Examples. This construct includes, in 5′ to 3′ order, the rat insulin promoter operably linked to a nucleic acid molecule encoding human Pdx1 by a chimeric intron, followed by a P2A linker, followed by a nucleic acid molecule encoding human MafA, followed by a T2A linker, followed by a nucleic acid encoding green fluorescent peptide, followed by a woodchuck hepatitis virus post-transcriptional regulatory element (WPRE), followed by a poly A tail.

FIGS. 4A-4G. RNAscope for the GFP RNA combined with immunostaining for insulin and glucagon in a pancreas infused with either (A) AAV-CMV-PM-GFP or (B) AAV-RIP-PM-GFP. Scale bar is 100 μm. C) Comparison between mouse weights in the four groups at 4 weeks, showing that the virus does not affect weight gain. (D) IPGTT at 4 weeks shows normalization of the glucose tolerance curve, with (E) showing the area under the curve (AUC). (F) Glucose excursion at very early time points during the in vivo GSIS. (G) Insulin levels from the experiment in (F) again showing the markedly improved first phase insulin secretion. Statistical analysis was done by One-way ANOVA (C,E) and two-way ANOVA (F,G) followed by Holm Sidak's test for multiple comparisons. * p<0.05, ** p<0.01, *** p<0.001, **** p<0.0001, and ns=no significance.

FIGS. 5A-5C. At five months after virus infusion using the rat insulin promoter (RIP) promoter, the animals are still euglycemic. The body weight remains high (A) as they have been maintained on the high fat diet, but despite that the glucose tolerance remains equivalent to animals maintained on a regular chow diet. AUC (B) and glucose tolerance testing curve (C).

### DETAILED DESCRIPTION OF SEVERAL EMBODIMENTS

It is disclosed herein that adeno-associated virus including an insulin promoter operably linked to nucleic acid molecules encoding Pdx1 and MafA can be used to treat T2DM in a subject, such as to improve hyperglucagonemia, improve insulin sensitivity, and/or improve glucose homeostasis in the subject. The adeno-associated virus can be infused through the pancreatic duct, such as by using ERCP, for the treatment of T2DM. Compositions are discussed that can be used in a subject with T2DM, such as to improve hyperglucagonemia, improve insulin sensitivity, and/or improve glucose homeostasis.

### Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a vector” includes singular or plural vectors and can be considered equivalent to the phrase “at least one vector.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Alpha (α) cells: Mature glucagon producing endocrine cells. In vivo, these cells are found in the pancreatic islets of Langerhans.

Beta (β) cells: Mature insulin producing endocrine cells. In vivo, these cells are found in the pancreatic islets of Langerhans.

Delta (δ) cells: Mature somatostatin producing endocrine cells. In vivo, these cells are found in the pancreatic islets of Langerhans.

PP cells: Mature pancreatic polypeptide (PP) producing endocrine cells. In vivo, these cells are found in the pancreatic islets of Langerhans.

Adeno-associated virus (AAV): A small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. There are currently 11 recognized serotypes of AAV (AAV1-11).

Administration: To provide or give a subject an agent by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, intraductal, vaginal and inhalation routes. In some aspects, administration is to a pancreatic duct.

Agent: Any polypeptide, compound, small molecule, organic compound, salt, polynucleotide, or other molecule of interest. Agent can include a therapeutic agent, a diagnostic agent or a pharmaceutical agent. A therapeutic agent is a substance that demonstrates some therapeutic effect by restoring or maintaining health, such as by alleviating the symptoms associated with a disease or physiological disorder, or delaying (including preventing) progression or onset of a disease, such as T2D. An agent can be an AAV vector encoding Pdx1 and MAFA.

Animal: Living multi-cellular vertebrate organisms, a category that includes, for example, mammals and birds. The term mammal includes both human and non-human mammals. Similarly, the term “subject” includes both human and veterinary subjects.

Anti-diabetic lifestyle modifications: Changes to lifestyle, habits, and practices intended to alleviate the symptoms of diabetes or pre-diabetes. Obesity and sedentary lifestyle may both independently increase the risk of a subject developing type II diabetes, so anti-diabetic lifestyle modifications include those changes that will lead to a reduction in a subject's body mass index (BMI), increase physical activity, or both. Specific, non-limiting examples include the lifestyle interventions described in Diabetes Care, 22(4):623-34 at pages 626-27, herein incorporated by reference.

Conservative Substitutions: Modifications of a polypeptide that involve the substitution of one or more amino acids for amino acids having similar biochemical properties that do not result in change or loss of a biological or biochemical function of the polypeptide are designated “conservative” substitutions. These conservative substitutions are likely to have minimal impact on the activity of the resultant protein. Table 1 shows amino acids that can be substituted for an original amino acid in a protein, and which are regarded as conservative substitutions.

TABLE

| Original Residue | Conservative Substitutions |
|---|---|
| Ala | ser |
| Arg | lys |
| Asn | gln; his |
| Asp | glu |
| Cys | ser |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

One or more conservative changes, or up to ten conservative changes (such as two substituted amino acids, three substituted amino acids, four substituted amino acids, or five substituted amino acids, etc.) can be made in the polypeptide without changing a biochemical function of the protein, such as Pdx1 or MafA.

Diabetes mellitus: A group of metabolic diseases in which a subject has high blood sugar, either because the pancreas does not produce enough insulin, or because cells do not respond to the insulin that is produced. Type 1 diabetes results from the body's failure to produce insulin. This form has also been called “insulin-dependent diabetes mellitus” (IDDM) or “juvenile diabetes”. Type 1 diabetes mellitus is characterized by loss of the insulin-producing βcells, leading to insulin deficiency. This type can be further classified as immune-mediated or idiopathic. Type 2 diabetes results from insulin resistance, a condition in which cells fail to use insulin properly, sometimes combined with an absolute insulin deficiency. This form is also called “non-insulin-dependent diabetes mellitus” (NIDDM) or “adult-onset diabetes.” The defective responsiveness of body tissues to insulin is believed to involve the insulin receptor. Diabetes mellitus is characterized by recurrent or persistent hyperglycemia, and is diagnosed by demonstrating any one of:
a. Fasting plasma glucose level≥7.0 mmol/l (126 mg/dl);
b. Plasma glucose≥11.1 mmol/l (200 mg/dL) two hours after a 75 g oral glucose load as in a glucose tolerance test;
c. Symptoms of hyperglycemia and casual plasma glucose≥11.1 mmol/l (200 mg/dl);
d. Glycated hemoglobin (Hb A1C)≥6.5%

Differentiation: The process whereby a first cell acquires specialized structural and/or functional features characteristic of a certain type of mature cells. Similarly, “differentiate” refers to this process. Typically, during differentiation, cellular structure alters and tissue-specific proteins appear. The term “differentiated pancreatic endocrine cell” refers to cells expressing a protein characteristic of the specific pancreatic endocrine cell type. A differentiated pancreatic endocrine cell includes an a cell, a β cell, a 8 cell, and a PP cell, which express glucagon, insulin, somatostatin, and pancreatic polypeptide, respectively.

Endocrine: Tissue which secretes regulatory hormones directly into the bloodstream without the need for an associated duct system.

Enhancer: A nucleic acid sequence that increases the rate of transcription by increasing the activity of a promoter.

Expand: A process by which the number or amount of cells is increased due to cell division. Similarly, the terms “expansion” or “expanded” refers to this process. The terms “proliferate,” “proliferation” or “proliferated” may be used interchangeably with the words “expand,” “expansion,” or “expanded.”

Expressed: Translation of a nucleic acid into a protein. Proteins may be expressed and remain intracellular, become a component of the cell surface membrane, or be secreted into the extracellular matrix or medium.

Exocrine: Secretory tissue which distributes its products, such as enzymes, via an associated duct network. The exocrine pancreas is the part of the pancreas that secretes enzymes required for digestion. The exocrine cells of the pancreas include the centroacinar cells and basophilic cells, which produce secretin and cholecystokinin.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Glucose homeostasis: The balance of insulin and glucagon to maintain blood glucose. Through hormones, particularly glucagon and insulin, the pancreas maintains blood glucose levels in humans within a very narrow range of 4-6 mm. As disclosed in Rorder et al., Exp. Mol. Med. 48(3): e219, 2016, preservation of blood glucose is accomplished by the opposing and balanced actions of glucagon and insulin. During sleep or in between meals, when blood glucose levels are low, glucagon is released from α-cells to promote hepatic glycogenolysis. In addition, glucagon drives hepatic and renal gluconeogenesis to increase endogenous blood glucose levels during prolonged fasting. In contrast, insulin secretion from β-cells is stimulated by elevated exogenous glucose levels, such as those occurring after a meal. After docking to its receptor on muscle and adipose tissue, insulin enables the insulin-dependent uptake of glucose into these tissues and hence lowers blood glucose levels by removing the exogenous glucose from the blood stream. Insulin promotes glycogenesis, lipogenesis and the incorporation of amino acids into proteins, and is an anabolic hormone, in contrast to the catabolic activity of glucagon.

Glucagon: A pancreatic hormone produced by the pancreatic a cells in vivo. Examples of glucagon amino acid sequences are shown in GENBANK® accession Nos: NP_002045.1 (pro-protein) (human); NP_032126.1 (mouse), both incorporated by reference. The term Glucagon also encompasses species variants, homologues, allelic forms, mutant forms, and equivalents thereof, including conservative substitutions, additions, deletions therein not adversely affecting the function, such as binding to the glucagon receptor. Glucagon is encoded by nucleic acid corresponding to GENBANK® Accession No: NM_002054.2 (human); NM_008100.3 (mouse), both incorporated by reference. The glucagon protein encoded by the glucagon is gene is a preproprotein that is cleaved into four distinct mature peptides. One of these, glucagon, is a pancreatic hormone that counteracts the glucose-lowering action of insulin by stimulating glycogenolysis and gluconeogenesis. Glucagon is a ligand for a specific G-protein linked receptor whose signaling pathway controls cell proliferation.

Heterologous: A heterologous sequence is a sequence that is not normally (in the wild-type sequence) found adjacent to a second sequence. In one aspect, the sequence is from a different genetic source, such as a virus or organism, than the second sequence.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Hyperglucagonemia: A state of excess glucagon secretion. In healthy individuals, insulin has a suppressive effect on alpha-cell function and on glucagon secretion. The most common cause of hyperglucagonemia is an absence or deficiency of the restraining influence of insulin on glucagon production. Hyperglucagonemia is observed in most, but not all, subjects with Type 2 diabetes during fasting. A “normal” human blood glucagon level is generally about 50 to 100 μg/mL Hyperglucagonemia is a blood glucagon level of greater than about 100 μg/mL.

Insulin: A protein hormone involved in the regulation of blood sugar levels that is produced by pancreatic beta cells. In vivo, insulin is produced as a precursor proinsulin, consisting of the B and A chains of insulin linked together via a connecting C-peptide. Insulin itself includes only the B and A chains. Exemplary insulin sequences are provided in GENBANK® Accession NO. NM_000207.2 (human) and NM_008386.3 (mouse), as available on Apr. 1, 2015, and are incorporated by reference herein. Exemplary nucleic acid sequences encoding insulin are provided in GENBANK® Accession No: NM_000207.2 (human) and NM_008386.3 (mouse), as available on Apr. 1, 2015, and are incorporated by reference herein. The term insulin also encompasses species variants, homologues, allelic forms, mutant forms, and equivalents thereof, including conservative substitutions, additions, deletions therein not adversely affecting the structure of function.

Insulin sensitivity: A determination of how sensitive the cells in the body are to insulin. “Insulin resistance” is impaired sensitivity of the cells of the body to diabetes, and is a component of T2DM. The fasting plasma glucose (FPG) test or the A1C test can be used to diagnose insulin resistance, such as in a subject with pre-diabetes. In humans, exemplary test results showing prediabetes (insulin resistance) are: A1C—about 5.7 to about 6.4 percent; FPG—about 100 to about 125 mg/dL (milligrams per deciliter), and/or OGTT—about 140 to about 199 mg/dL Signs of insulin resistance include a waistline over about 40 inches in men and over about 35 inches in women; blood pressure readings of about 130/80 or higher; a fasting glucose level over about 100 mg/d; a fasting triglyceride level over about 150 mg/dL; a HDL cholesterol level under about 40 mg/dL in men and about 50 mg/dL in women, skin tags, and patches of dark, velvety skin (acanthosis nigricans).

Islets of Langerhans: Small discrete clusters of pancreatic endocrine tissue. In vivo, in an adult mammal, the islets of Langerhans are found in the pancreas as discrete clusters (islands) of pancreatic endocrine tissue surrounded by the pancreatic exocrine (or acinar) tissue. In vivo, the islets of Langerhans consist of the α cells, β cells, δ cells, PP cells, and ε cells. Histologically, in rodents, the islets of Langerhans consist of a central core of β cells surrounded by an outer layer of α cells, δ cells, and PP cells. The structure of human islets of Langerhans is different and distinct from rodents. The islets of Langerhans are sometimes referred to herein as “islets.”

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. An isolated cell type has been substantially separated from other cell types, such as a different cell type that occurs in an organ. A purified cell or component can be at least 90%, at least 95%, at least 96%, at least 97%, at least 98%, or at least 99% pure.

Label: A detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes.

Musculoaponeurotic fibrosarcoma oncogene homolog A (MafA): MAFA is a transcription factor that binds RIPE3b, a conserved enhancer element that regulates pancreatic beta cell-specific expression of the insulin gene (INS; MIM 176730) (Olbrot et al., 2002). MafA is referred in the art as aliases; v-maf musculoaponeurotic fibrosarcoma oncogene homolog A (avian), hMafA; RIPE3b1; MAFA. Examplary MafA proteins are teh MafA protein of GENBANK® Accession No: NM_194350 (mouse) (SEQ ID NO:3 32 of U.S. Published Patent Application No. 2011/0280842) or NP_963883.2 (Human) (SEQ ID NOs: 33 and 32 of U.S. Published Patent Application No. 2011/0280842); GeneID No: 389692, which are all incorporated by reference. The term MafA also encompasses species variants, homologues, allelic forms, mutant forms, and equivalents thereof, including conservative substitutions, additions, deletions that do not adversely affecting the structure of function. The term “MafA”, or “MafA” protein” as used herein refers to a polypeptide having a naturally occurring amino acid sequence of a MafA” protein or a fragment, variant, or derivative thereof retains the ability of the naturally occurring protein to bind to DNA and activate gene transcription of Glut2 and pyruvate carboxylase, and other genes such as Glut2, Pdx-1, Nkx6.1, GLP-1 receptor, prohormone convertase-1/3 as disclosed in Wang et al., Diabetologia. 2007 February; 50(2): 348-358, which is incorporated herein by reference. Exemplary MafA nucleci acids are GENBANK® Accession No: NM_201589 (human) (SEQ ID NO:36 32 of U.S. Published Patent Application No. 2011/0280842) and GENBANK® Accession No: NM_194350 (mouse) (SEQ ID NO: 39 32 of U.S. Published Patent Application No. 2011/0280842), which are all incorporated by reference. In addition to naturally-occurring allelic variants of the MafA sequences that may exist in the population, it will be appreciated that, as is the case for virtually all proteins, a variety of changes can be introduced into the sequences of SEQ ID NO: 3 32 of U.S. Published Patent Application No. 2011/0280842 or SEQ ID NO: 33 32 of U.S. Published Patent Application No. 2011/0280842 (referred to as “wild type” sequences) without substantially altering the functional (biological) activity of the polypeptides. Such variants are included within the scope of the terms “MafA”, “MafA protein”, etc. U.S. Published Patent Application No. 2011/0280842 and all of the GENBANK entries are incorporated herein by reference.

Mammal: This term includes both human and non-human mammals. Similarly, the term “subject” includes both human and veterinary subjects.

Neurogenin (Ngn) 3: Neurogenin-3 (also known as NEUROG3) is expressed in endocrine progenitor cells and is required for endocrine cell development in the pancreas and intestine. It belongs to a family of basic helix-loop-helix transcription factors involved in the determination of neural precursor cells in the neuroectoderm. Ngn3 is referred in the art as aliases; Neurogenin 3; Atoh5; Math4B; bHLHa7; NEUROG3. Exemplary Ngn3 proteins are provided in GENBANK® Accession No: NM_009719 (mouse) and SEQ ID NO:2 of U.S. Published Patent Application No. 2011/0280842, both incorporated by reference herein or GENBANK® Accession No: NP_033849.3 (Human) and SEQ ID NO: 32 of U.S. Published Patent Application No. 2011/0280842, both incorporated by reference herein; GeneID No: 50674. The term Ngn3 also encompasses species variants, homologues, allelic forms, mutant forms, and equivalents thereof, including conservative substitutions, additions, deletions therein not adversely affecting the structure of function. Human Ngn3 is encoded by nucleic acid corresponding to GENBANK® Accession No: NM_020999 (human), SEQ ID NO:35 of U.S. Published Patent Application No. 2011/0280842 or NM_009719 (mouse), SEQ ID NO: 38 of U.S. Published Patent Application No. 2011/0280842. U.S. Published Patent Application No. 2011/0280842 and these GENBANK® Accession Nos. are incorporated by reference herein. The term “Ngn3”, or “Ngn3 protein” as used herein refers to a polypeptide having a naturally occurring amino acid sequence of a Ngn3 protein or a fragment, variant, or derivative thereof that retains the ability of the naturally occurring protein to bind to DNA and activate gene transcription of NeuroD, Delta-like 1 (DI11), HeyL, insulinoma-associated-1 (IA1), Nk2.2, Notch, HesS, Isl1, Somatostatin receptor 2 (Sstr2) and other genes as disclosed in Serafimidis et al., Stem cells; 2008; 26; 3-16, which is incorporated herein in its entirety by reference. In addition to naturally-occurring allelic variants of the Ngn3 sequences that may exist in the population, it will be appreciated that, as is the case for virtually all proteins, a variety of changes can be introduced into a wild-type sequence (listed above in GENBANK® enteries) without substantially altering the functional (biological) activity of the polypeptides. Such variants are included within the scope of the terms “Ngn3”, “Ngn3 protein”, etc.

Nucleic acid: A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together. This includes nucleic acid vectors comprising an amplified or assembled nucleic acid which can be used to transform a suitable host cell. A host cell that comprises the recombinant nucleic acid is referred to as a “recombinant host cell.” The gene is then expressed in the recombinant host cell to produce, such as a “recombinant polypeptide.” A recombinant nucleic acid may serve a non-coding function (such as a promoter, origin of replication, ribosome-binding site, etc.) as well.

A first sequence is an “antisense” with respect to a second sequence if a polynucleotide whose sequence is the first sequence specifically hybridizes with a polynucleotide whose sequence is the second sequence.

Terms used to describe sequence relationships between two or more nucleotide sequences or amino acid sequences include “reference sequence,” “selected from,” “comparison window,” “identical,” “percentage of sequence identity,” “substantially identical,” “complementary,” and “substantially complementary.”

For sequence comparison of nucleic acid sequences, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are used. Methods of alignment of sequences for comparison are well known in the art. Optimal alignment of sequences for comparison can be conducted, for example, by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of Pearson & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see for example, Current Protocols in Molecular Biology (Ausubel et al., eds 1995 supplement)).

One example of a useful algorithm is PILEUP. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, such as version 7.0 (Devereaux et al., Nuc. Acids Res. 12:387-395, 1984.

Another example of algorithms that are suitable for determining percent sequence identity and sequence similarity are the BLAST and the BLAST 2.0 algorithm, which are described in Altschul et al., J. Mol. Biol. 215:403-410, 1990 and Altschul et al., Nucleic Acids Res. 25:3389-3402, 1977. Software for performing BLAST analyses is publicly available through the National Center for Biotechnology Information (http://www.ncbi.nlm.nih.gov/). The BLASTN program (for nucleotide sequences) uses as defaults a word length (W) of 11, alignments (B) of 50, expectation (E) of 10, M=5, N=−4, and a comparison of both strands. The BLASTP program (for amino acid sequences) uses as defaults a word length (W) of 3, and expectation (E) of 10, and the BLOSUM62 scoring matrix (see Henikoff & Henikoff, Proc. Natl. Acad. Sci. USA 89:10915, 1989).

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

ORF (open reading frame): A series of nucleotide triplets (codons) coding for amino acids without any termination codons. These sequences are usually translatable into a peptide.

Pancreatic endocrine cell: An endocrine cell of pancreatic origin that produces one or more pancreatic hormone, such as insulin, glucagon, somatostatin, or pancreatic polypeptide. Subsets of pancreatic endocrine cells include the α (glucagon producing), β (insulin producing) δ (somatostatin producing) or PP (pancreatic polypeptide producing) cells. Additional subsets produce more than one pancreatic hormone, such as, but not limited to, a cell that produces both insulin and glucagon, or a cell that produces insulin, glucagon, and somatostatin, or a cell that produces insulin and somatostatin.

Pancreas duodenal homeobox protein (Pdx) 1: Pdx1 protein is a transcriptional activator of several genes, including insulin, somatostatin, glucokinase, islet amyloid polypeptide, and glucose transporter type 2 (GLUT2). Pdx1 is a nuclear protein is involved in the early development of the pancreas and plays a major role in glucose-dependent regulation of insulin gene expression. Defects in the gene encoding the Pdx1 protein are a cause of pancreatic agenesis, which can lead to early-onset insulin-dependent diabetes mellitus (NIDDM), as well as maturity onset diabetes of the young type 4 (MODY4). Pdx1 is referred in the art as aliases; pancreatic and duodenal homeobox 1, IDX-1, STF-1, PDX-1, MODY4, Ipfl. Exemplary Pdx1 proteins are shown in GENBANK® Accession No. NM_008814 (mouse) (SEQ ID NO: 1 of U.S. Published Patent Application No. 2011/0280842) or GENBANK® Accession No. NP_000200.1 (Human) (SEQ ID NO: 31 of U.S. Published Patent Application No. 2011/0280842), or Gene ID: 3651, which are all incorporated herein by reference. The term Pdx1 also encompasses species variants, homologues, allelic forms, mutant forms, and equivalents thereof, including conservative substitutions, additions, deletions therein not adversely affecting the structure of function. Exemplary nucleic acid sequences are shown in GENBANK® Accession No NM_000209 (human) (SEQ ID NO:34 of U.S. Published Patent Application No. 2011/0280842) or GENBANK® Accession No NM_008814 (mouse) (SEQ ID NO: 37 of U.S. Published Patent Application No. 2011/0280842), which are all incorporated by reference. The term “Pdx1”, or “Pdx1 protein” as used herein refers to a polypeptide having a naturally occurring amino acid sequence of a Pdx1 protein or a fragment, variant, or derivative thereof that at least in part retains the ability of the naturally occurring protein to bind to DNA and activate gene transcription of insulin, somatostatin, glucokinase, islet amyloid polypeptide, and glucose transporter type 2 (GLUT2). In addition to naturally-occurring allelic variants of the Pdx1 sequences that may exist in the population, it will be appreciated that, as is the case for virtually all proteins, a variety of changes can be introduced into a wild type sequence (see the listed GENBANK® entries) without substantially altering the functional (biological) activity of the polypeptides. Such variants are included within the scope of the terms “Pdx1”, “Pdx1 protein”, etc. The listed GENBANK® Accession Nos. and of U.S. Published Patent Application No. 2011/0280842 are incorporated by reference herein.

Pharmaceutically acceptable carriers: The pharmaceutically acceptable carriers useful with the disclosed methods are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or a composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Pre-diabetes: A state in which some, but not all, of the criteria for diabetes are met. For example, a subject can have impaired fasting glycaemia or impaired fasting glucose (IFG). Subjects with fasting glucose levels from 110 to 125 mg/dl (6.1 to 6.9 mmol/l) are considered to have impaired fasting glucose. Subjects with plasma glucose at or above 140 mg/dL (7.8 mmol/L), but not over 200 mg/dL (11.1 mmol/L), two hours after a 75 g oral glucose load are considered to have impaired glucose tolerance. Predisposition for diabetes: A subject that is at high risk for developing diabetes. A number of risk factors are known to those of skill in the art and include: genetic factors (e.g., carrying alleles that result in a higher occurrence of diabetes than in the average population or having parents or siblings with diabetes); overweight (e.g., body mass index (BMI) greater or equal to 25 kg/m.sup.2); habitual physical inactivity, race/ethnicity (e.g., African-American, Hispanic-American, Native Americans, Asian-Americans, Pacific Islanders); previously identified impaired fasting glucose or impaired glucose tolerance, hypertension (e.g., greater or equal to 140/90 mmHg in adults); HDL cholesterol greater or equal to 35 mg/dl; triglyceride levels greater or equal to 250 mg/dl; a history of gestational diabetes or delivery of a baby over nine pounds; and/or polycystic ovary syndrome. See, e.g., “Report of the Expert Committee on the Diagnosis and Classification of Diabetes Mellitus” and “Screening for Diabetes” Diabetes Care 25(1): S5-S24 (2002).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein is intended to encompass any amino acid sequence and include modified sequences such as glycoproteins. The term “polypeptide” is specifically intended to cover naturally occurring proteins, as well as those that are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “substantially purified polypeptide” as used herein refers to a polypeptide which is substantially free of other proteins, lipids, carbohydrates or other materials with which it is naturally associated. In one aspect, the polypeptide is at least 50%, for example at least 80% free of other proteins, lipids, carbohydrates or other materials with which it is naturally associated. In another aspect, the polypeptide is at least 90% free of other proteins, lipids, carbohydrates or other materials with which it is naturally associated. In yet another aspect, the polypeptide is at least 95% free of other proteins, lipids, carbohydrates or other materials with which it is naturally associated.

Conservative substitutions replace one amino acid with another amino acid that is similar in size, hydrophobicity, etc. Variations in the cDNA sequence that result in amino acid changes, whether conservative or not, should be minimized in order to preserve the functional and immunologic identity of the encoded protein. The immunologic identity of the protein may be assessed by determining if it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may, for example, be 80, 90 or even 95% or 98% identical to the native amino acid sequence.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Preventing, treating or ameliorating a disease: “Preventing” a disease (such as T2DM) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see for example, Bitter et al., Methods in Enzymology 153:516-544, 1987).

Purified: The term “purified” does not require absolute purity; rather, it is intended as a relative term. Thus, for example, a purified peptide, protein, virus, or other active compound is one that is isolated in whole or in part from naturally associated proteins and other contaminants. In certain aspects, the term “substantially purified” refers to a peptide, protein, virus or other active compound that has been isolated from a cell, cell culture medium, or other crude preparation and subjected to fractionation to remove various components of the initial preparation, such as proteins, cellular debris, and other components.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, such as by genetic engineering techniques. Similarly, a recombinant protein is one encoded for by a recombinant nucleic acid molecule. In addition, a recombinant virus is a virus comprising sequence (such as genomic sequence) that is non-naturally occurring or made by artificial combination of at least two sequences of different origin. The term “recombinant” also includes nucleic acids, proteins and viruses that have been altered solely by addition, substitution, or deletion of a portion of a natural nucleic acid molecule, protein or virus. As used herein, “recombinant AAV” refers to an AAV particle in which a recombinant nucleic acid molecule (such as a recombinant nucleic acid molecule encoding Pdx1 and MafA) has been packaged.

Selectable Marker: A gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select specific cells of interest. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. The term “selectable marker” as used herein can refer to a gene or to an expression product of the gene, e.g., an encoded protein.

Revitalize: In Type 2 diabetes many of the beta cells have become senescent and poorly functioning. The intervention proposed here involves these poorly functioning cells regaining their better function, or becoming revitalized.

Sequence identity of amino acid sequences: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Homologs and variants of proteins, such as MafA or Pdx1 are typically characterized by possession of at least about 75%, for example at least about 80%, 90%, 95%, 96%, 97%, 98% or 99% sequence identity counted over the full length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Specific binding agent: An agent that binds substantially only to a defined target. Thus a β cell specific binding agent is an agent that binds substantially to a β cell, and a pancreatic endocrine cell specific binding agent is an gent that binds substantially only to pancreatic endocrine cells or a subset thereof (and not to pancreatic exocrine cells). Similarly, a pancreatic exocrine cell specific binding agent is an agent that binds substantially to exocrine cells. In one aspect, the specific binding agent is a monoclonal or polyclonal antibody that specifically binds a type of pancreatic cell.

The term “specifically binds” refers, with respect to a cell, such as a pancreatic endocrine cell, to the preferential association of an antibody or other ligand, in whole or part, with a cell or tissue bearing that antigen and not to cells or tissues lacking that antigen. It is, of course, recognized that a certain degree of non-specific interaction may occur between a molecule and a non-target cell or tissue. Nevertheless, specific binding may be distinguished as mediated through specific recognition of the antigen. Although selectively reactive antibodies bind antigen, they may do so with low affinity. On the other hand, specific binding results in a much stronger association between the antibody (or other ligand) and cells bearing the antigen than between the bound antibody (or other ligand) and cells lacking the antigen. Specific binding typically results in greater than 2-fold, such as greater than 5-fold, greater than 10-fold, or greater than 100-fold increase in amount of bound antibody or other ligand (per unit time) to a cell or tissue expressing the target epitope as compared to a cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Subject: Any mammal, such as humans, non-human primates, pigs, sheep, cows, rodents and the like which is to be the recipient of the particular treatment. In two non-limiting examples, a subject is a human subject or a murine subject.

Therapeutic agent: Used in a generic sense, it includes treating agents, prophylactic agents, and replacement agents. A therapeutic agent can be a nucleic acid molecule encoding MafA and Pdx-1, or a vector encoding these factors.

Therapeutically effective amount: A quantity of a specified pharmaceutical or therapeutic agent (e.g. a recombinant AAV) sufficient to achieve a desired effect in a subject, or in a cell, being treated with the agent, such as increasing insulin production. The effective amount of the agent will be dependent on several factors, including, but not limited to the subject or cells being treated, and the manner of administration of the therapeutic composition.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses {Wolff, J. A., ed, Gene Therapeutics, Birkhauser, Boston, USA (1994)}. In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an artificial islet produced by the methods described herein.

Genetic modification of the target cell is an indicium of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like. In some aspects herein, the vector is an AAV vector.

It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

### Overview of Several Aspects

Disclosed herein are methods for treating a subject with T2DM. These methods include administering to the subject a therapeutically effective amount of a vector including an insulin promoter operably linked to a nucleic acid molecule encoding heterologous Pdx1 and MafA. In some aspects, the vector does not encode Ngn3, and the subject is not administered any other nucleic acid encoding Ngn3. In some aspects, the vector is administered intraductally into a pancreatic duct of the subject. In some aspects, the disclosed methods revitalize pancreatic beta cells in the subject. The disclosed methods can improve hyperglucagonemia, improve insulin sensitivity, and/or improve glucose homeostasis in the subject. The method can include selecting a subject with T2DM.

In additional aspects, the vector is an adenovirus vector or an adeno-associated virus vector, such as, but not limited to, an adeno-associated virus 8 vector. The subject can be a human or a veterinary subject. In some aspects, the subject has T2DM. In more aspects, the subject is also administered metformin.

In some aspects, the vector, such as the adenovirus vector or adeno-associated virus vector includes a human insulin promoter including, or consisting of, the nucleic acid sequence of:

```
  (SEQ ID NO: 1)
TCCTGAGGAAGAGGTGCTGACGACCAAGGAGATCTTCCCACAGACCC
AGCACCAGGGAAATGGTCCGGAAATTGCAGCCTCAGCCCCCAGCCATCTGCCGACCCCCCCAC
CCCAGGCCCTAATGGGCCAGGCGGCAGGGGTTGAGAGGTAGGGGAGATGGGCTCTGAGACTA
TAAAGCCAGCGGGGGCCCAGCAGCCCTCAGCCCTCCAGGACAGGCTGCATCAGAAGAGGCCAT
CAAGCAGGTCTGTTCC
or
  (SEQ ID NO: 2)
GAGCTCTGTGGGGACAGGGGTCTGGGGACAGCAGCGCAAAGAGCCCCGCCCTGCAGCCTCCAG
CTCTCCTGGTCTAATGTGGAAAGTGGCCCAGGTGAGGGCTTTGCTCTCCTGGAGACATTTGCCC
CCAGCTGTGAGCAGGGACAGGTCTGGCCACCGGGCCCCTGGTTAAGACTCTAATGACCCGCTG
GTCCTGAGGAAGAGGTGCTGACGACCAAGGAGATCTTCCCACAGACCCAGCACCAGGGAAAT
GGTCCGGAAATTGCAGCCTCAGCCCCCAGCCATCTGCCGACCCCCCCACCCCAGGCCCTAATG
GGCCAGGCGGCAGGGGTTGACAGGTAGGGGAGATGGGCTCTGAGACTATAAAGCCAGCGGGG
GCCCAGCAGCCCTCAGCCCTCCAGGACAGGCTGCATCAGAAGCTT.
```

In other aspects, the vector, such as the adenovirus vector or adeno-associated virus vector includes a rat insulin promoter including, or consisting of, the nucleic acid sequence of:

(SEQ ID NO: 3)
CAAGTGGAGGCTGAGAAAGGTTTTGTAGCTGGGTAGAGTATGTACTAAGAGATGGAGACAGCT
GGCTCTGAGCTCTGAAGCAAGCACCTCTTATGGAGAGTTGCTGACCTTCAGGTGCAAATCTAAG
ATACTACAGGAGAATACACCATGGGGCTTCAGCCCAGTTGACTCCCGAGTGGGCTATGGGTTT
GTGGAAGGAGAGATAGAAGAGAAGGGACCTTTCTTCTTGAATTCTGCTTTCCTTCTACCTCTGA
GGGTGAGCTGGGGTCTCAGCTGAGGTGAGGACACAGCTATCAGTGGGAACTGTGAAACAACA
GTTCAAGGGACAAAGTTACTAGGTCCCCCAACAACTGCAGCCTCCTGGGGAATGATGTGGAAA
AATGCTCAGCCAAGGACAAAGAAGGCCTCACCCTCTCTGAGACAATGTCCCCTGCTGTGAACT
GGTTCATCAGGCCACCCAGGAGCCCCTATTAAGACTCTAATTACCCTAAGGCTAAGTAGAGGT
GTTGTTGTCCAATGAGCACTTTCTGCAGACCTAGCACCAGGCAAGTGTTTGGAAACTGCAGCTT
CAGCCCCTCTGGCCATCTGCTGATCCACCCTTAATGGGACAAACAGCAAAGTCCAGGGGTCAG
GGGGGGGGTGCTTTGGACTATAAAGCTAGTGGGGATTCAGTAACCCCCAGCCCTAA

In additional aspects, the adenovirus vector or the adeno-associated virus vector comprises the promoter operably linked to the nucleic acid sequence encoding Pdx1 and the nucleic acid sequence encoding MaFA, wherein the nucleic acid sequence encoding Pdx1 and the nucleic acid sequence encoding MafA are linked using a connector. An exemplary connector is a 2A connector. The adenovirus or adeno-associated virus vector also can include a nucleic acid sequence encoding a label.

In further aspects, the vector, such as the adenovirus vector is administered intraductally using endoscopic retrograde cholangiopancreatography (ERCP).

In yet other aspects, the method improves hyperglucagonemia, insulin sensitivity, and/or glucose homeostasis in the subject as compared to a control value, wherein the control value is hyperglucagonemia, insulin sensitivity, and/or glucose homeostasis, respectively, in the subject prior to treatment with the vector. Compositions for use in any of the methods disclosed herein also are provided. In some aspects, these compositions include a) a adeno-associated virus vector comprising an insulin promoter operably linked to a nucleic acids encoding Pdx1 and a nucleic acid encoding MafA, b) a pharmaceutically acceptable carrier; and optionally c) a contrast dye for endoscopic retrograde cholangiopancreatography. In specific non-limiting examples, the dye is included in the composition. In some aspects, these compositions does not include a nucleic acid encoding Ngn3 (either in the vector or as a separate nucleic acid) and do not include a Ngn3 polypeptide. In some aspects, the promoter in the adeno-associated virus vector or adenovirus vector includes or consists of the nucleic acid sequence set forth as one of SEQ ID Nos: 1-3.

In additional non-limiting examples, the adeno-associated virus vector is an adeno-associated virus 8 vector. In some aspects, the nucleic acid sequence encoding Pdx1 and a nucleic acid sequence encoding MaFA are joined by a connector. The connector can be a 2A connector. The adeno-associated virus vector also can include a nucleic acid sequence encoding a label.

In some aspects, the contrast dye is a low-osmolar low-viscosity non-ionic dye, a low-viscosity high-osmolar dye, or a dissociable high-viscosity dye. Specific non-limiting examples of a contrast dye are Iopromid, Ioglicinate, and Ioxaglinate.

The composition can be formulated for administration to the pancreatic duct, for example by using endoscopic retrograde cholangiopancreatography (ERCP).

The composition including an adenovirus of adeno-associated virus vector comprising an insulin promoter operably linked to a nucleic acid encoding Pdx1 and a nucleic acid encoding MafA, wherein the vector is administered intraductally into a pancreatic duct of the subject, can be used for the treatment of T2DM. In some aspects, the composition improves hyperglucagonemia, insulin sensitivity, and/or glucose homeostasis in the subject. In more aspects, the composition does not include a nucleic acid encoding Ngn3 or Ngn3 polypeptide.

### Vectors

Disclosed herein are vectors, such as a viral vector, such as a retroviral vector, an adenoviral vector, or an adeno-associated vector (AAV) that include an insulin promoter operably linked to a nucleic acid sequence that encodes MafA and Pdx1. Viral vectors include an attenuated or defective DNA or RNA viruses, including, but not limited to, adenovirus or adeno-associated virus (AAV). Defective viruses, that entirely or almost entirely lack viral genes, can be used. Use of defective viral vectors allows for administration to specific cells without concern that the vector can infect other cells. In some examples, the vector is an attenuated adenovirus vector, such as the vector described by Stratford-Perricaudet et al. (J. Clin. Invest., 90:626-630 1992; La Salle et al., Science 259:988-990, 1993); and a defective adeno-associated virus vector (Samulski et al., J. Virol., 61:3096-3101, 1987; Samulski et al., J. Virol., 63:3822-3828, 1989; Lebkowski et al., Mol. Cell. Biol., 8:3988-3996, 1988).

Suitable vectors are known in the art, and include viral vectors such as retroviral, lentiviral, adenoviral vectors, and AAV. In specific, non-limiting examples, the vector is a lentiviral vector, gammaretroviral vector, self-inactivating retroviral vector, adenoviral vector, or adeno-associated vector (AAV).

Adenoviral vectors and/or adeno-associated viral vectors can be used in the methods disclosed herein. AAV belongs to the family Parvoviridae and the genus Dependovirus. AAV is a small, non-enveloped virus that packages a linear, single-stranded DNA genome. Both sense and antisense strands of AAV DNA are packaged into AAV capsids with equal frequency. In some aspects the AAV DNA includes a nucleic acid encoding Pdx1 and MafA, but does not include a nucleic acid encoding Ngn3. Further provided are recombinant vectors, such as recombinant adenovirus vectors and recombinant adeno-associated virus (rAAV) vectors comprising a nucleic acid molecule disclosed herein. In some aspects, the AAV is rAAV8 and/or AAV2. However, the AAV serotype can be any other suitable AAV serotype, such as AAV1, AAV2, AAV3, AAV4, AAV5, AAV6, AAV7, AAV9, AAV10, AAV11 or AAV12, or a hybrid of two or more AAV serotypes (such as, but not limited to AAV2/1, AAV2/7, AAV2/8 or AAV2/9).

The AAV genome is characterized by two inverted terminal repeats (ITRs) that flank two open reading frames (ORFs). In the AAV2 genome, for example, the first 125 nucleotides of the ITR are a palindrome, which folds upon itself to maximize base pairing and forms a T-shaped hairpin structure. The other 20 bases of the ITR, called the D sequence, remain unpaired. The ITRs are cis-acting sequences important for AAV DNA replication; the ITR is the origin of replication and serves as a primer for second-strand synthesis by DNA polymerase. The double-stranded DNA formed during this synthesis, which is called replicating-form monomer, is used for a second round of self-priming replication and forms a replicating-form dimer. These double-stranded intermediates are processed via a strand displacement mechanism, resulting in single-stranded DNA used for packaging and double-stranded DNA used for transcription. Located within the ITR are the Rep binding elements and a terminal resolution site (TRS). These features are used by the viral regulatory protein Rep during AAV replication to process the double-stranded intermediates. In addition to their role in AAV replication, the ITR is also essential for AAV genome packaging, transcription, negative regulation under non-permissive conditions, and site-specific integration (Daya and Berns, Clin Microbiol Rev 21(4):583-593, 2008). In some aspects, these elements are included in the AAV vector.

The left ORF of AAV contains the Rep gene, which encodes four proteins-Rep78, Rep 68, Rep52 and Rep40. The right ORF contains the Cap gene, which produces three viral capsid proteins (VP1, VP2 and VP3). The AAV capsid contains 60 viral capsid proteins arranged into an icosahedral symmetry. VP1, VP2 and VP3 are present in a 1:1:10 molar ratio (Daya and Berns, Clin Microbiol Rev 21(4):583-593, 2008). In some aspects, these elements are included in the AAV vector.

AAV vectors can be used for gene therapy. Exemplary AAV of use are AAV2, AAV5, AAV6, AAV8 and AAV9. Adenovirus, AAV2 and AAV8 are capable of transducing cells in the pancreas. Thus, any of a rAAV2 or rAAV8 vector can be used in the methods disclosed herein. However, rAAV6 and rAAV9 vectors are also of use.

Although AAV infects humans and some other primate species, it is not known to cause disease and elicits a very mild immune response. Gene therapy vectors that utilize AAV can infect both dividing and quiescent cells and persist in an extrachromosomal state without integrating into the genome of the host cell. AAV8 preferentially infects cells of the pancreas. Because of the advantageous features of AAV, the present disclosure contemplates the use of an rAAV for the methods disclosed herein.

AAV possesses several additional desirable features for a gene therapy vector, including the ability to bind and enter target cells, enter the nucleus, the ability to be expressed in the nucleus for a prolonged period of time, and low toxicity. AAV can be used to transfect cells, and suitable vector are known in the art, see for example, U.S. Published Patent Application No. 2014/0037585, incorporated herein by reference. Methods for producing rAAV suitable for gene therapy are well known in the art (see, for example, U.S. Published Patent Application Nos. 2012/0100606; 2012/0135515; 2011/0229971; and 2013/0072548; and Ghosh et al., Gene Ther 13(4):321-329, 2006), and can be utilized with the methods disclosed herein.

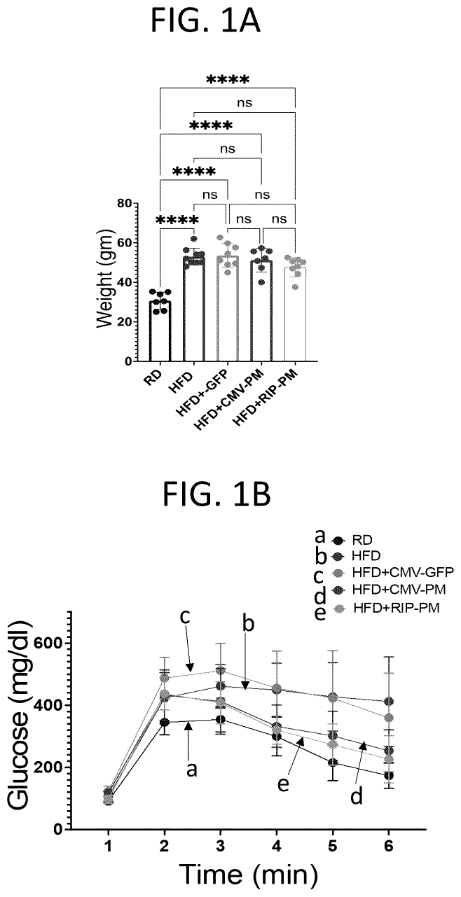

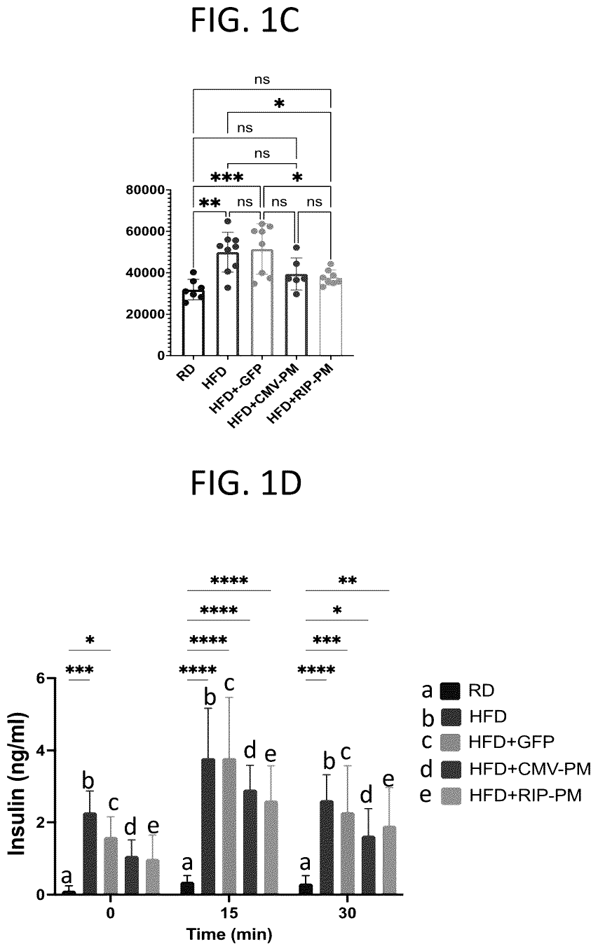

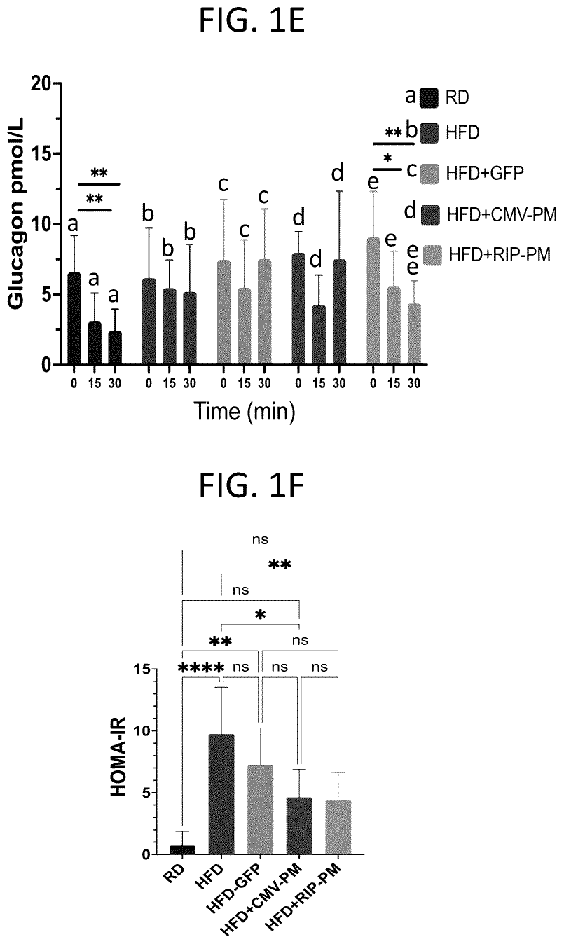

In some aspects, the vector is an rAAV8 vector, an rAAV6 vector, or an rAAV9 vector. AAV8 vectors are disclosed, for example, in U.S. Pat. No. 8,692,332, which is incorporated by reference herein. An exemplary AAV8 nucleic acid sequence is shown in FIG. 1 and SEQ ID NO: 1 of U.S. Pat. No. 8,692,332. It is disclosed that AAV nucleic acid sequence can be greater than about 90%, 95%, 98% or 99% identical to this nucleic acid sequence. The location and sequence of the capsid, rep 68/78, rep 40/52, VP1, VP2 and VP3 are disclosed in this U.S. Pat. No. 8,692,332. The location and hypervariable regions of AAV8 are also provided.

The vectors of use in the methods disclosed herein can contain nucleic acid sequences encoding an intact AAV capsid which may be from a single AAV serotype (e.g., AAV2, AAV, 6, AAV8 or AAV9). As disclosed in U.S. Pat. No. 8,692,332, vectors of use can also be recombinant, and thus can contain sequences encoding artificial capsids which contain one or more fragments of the AAV8 capsid fused to heterologous AAV or non-AAV capsid proteins (or fragments thereof). These artificial capsid proteins are selected from non-contiguous portions of the AAV2, AAV6, AAV8 or AAV9 capsid or from capsids of other AAV serotypes. For example, a rAAV vector may have a capsid protein comprising one or more of the AAV8 capsid regions selected from the VP2 and/or VP3, or from VP1, or fragments thereof selected from amino acids 1 to 184, amino acids 199 to 259; amino acids 274 to 446; amino acids 603 to 659; amino acids 670 to 706; amino acids 724 to 738 of the AAV8 capsid, see SEQ ID NO: 2 of U.S. Pat. No. 8,692,332. In another example, it may be desirable to alter the start codon of the VP3 protein to GTG. Alternatively, the rAAV may contain one or more of the AAV serotype 8 capsid protein hypervariable regions, for example aa 185-198; aa 260-273; aa447-477; aa495-602; aa660-669; and aa707-723 of the AAV8 capsid set forth in SEQ ID NO: 2 of U.S. Pat. No. 8,692,332.

In some aspects, a recombinant adeno-associated virus (rAAV) is generated having an AAV serotype 8 capsid. To produce the vector, a host cell which can be cultured that contains a nucleic acid sequence encoding an adeno-associated virus (AAV) serotype 8 capsid protein, or fragment thereof, as defined herein; a functional rep gene; a minigene composed of, at a minimum, AAV inverted terminal repeats (ITRs) and a transgene, such as a transgene encoding Pdx1 and MafA; and sufficient helper functions to permit packaging in the AAV8 capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art. In some aspects, a stable host cell will contain the required component(s) under the control of an inducible promoter. However, the required component(s) can be under the control of a constitutive promoter. Examples of suitable inducible and constitutive promoters are provided below. Similar methods can be used to generate a rAAV2, rAAV6 or rAAV9 vector and/or virion.

In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing a rAAV can be delivered to the packaging host cell in the form of any genetic element which transfer the sequences carried thereon. The selected genetic element may be delivered by any suitable method, including those described herein. The methods used to construct vectors are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745. In some aspects, selected AAV components can be readily isolated using techniques available to those of skill in the art from an AAV serotype, including AAV8. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GENBANK®.

The adenovirus and AAV vectors disclosed herein include a promoter operably linked to a nucleic acid encoding PDx1 and MafA. In some aspects, the adenovirus or AAV vector does not include a nucleic acid encoding Ngn3. In some aspects, the promoter is an insulin promoter, such as a human, rat or a mouse insulin promoter.

An exemplary human insulin promoter is:

(SEQ ID NO: 1)
TCCTGAGGAAGAGGTGCTGACGACCAAGGAGATCTTCCCACAGACCC
AGCACCAGGGAAATGGTCCGGAAATTGCAGCCTCAGCCCCCAGCCAT
CTGCCGACCCCCCCACCCCAGGCCCTAATGGGCCAGGCGGCAGGGGT
TGAGAGGTAGGGGAGATGGGCTCTGAGACTATAAAGCCAGCGGGGGC
CCAGCAGCCCTCAGCCCTCCAGGACAGGCTGCATCAGAAGAGGCCAT
CAAGCAGGTCTGTTCC.

Another exemplary human insulin promoter is:

(SEQ ID NO: 2)
GAGCTCTGTGGGGACAGGGGTCTGGGGACAGCAGCGCAAAGAGCCCCGC
CCTGCAGCCTCCAGCTCTCCTGGTCTAATGTGGAAAGTGGCCCAGGTGA
GGGCTTTGCTCTCCTGGAGACATTTGCCCCCAGCTGTGAGCAGGGACAG
GTCTGGCCACCGGGCCCCTGGTTAAGACTCTAATGACCCGCTGGTCCTG
AGGAAGAGGTGCTGACGACCAAGGAGATCTTCCCACAGACCCAGCACCA
GGGAAATGGTCCGGAAATTGCAGCCTCAGCCCCCAGCCATCTGCCGACC
CCCCCACCCCAGGCCCTAATGGGCCAGGCGGCAGGGGTTGACAGGTAGG
GGAGATGGGCTCTGAGACTATAAAGCCAGCGGGGGCCCAGCAGCCCTCA
GCCCTCCAGGACAGGCTGCATCAGAAGCTT.

One of skill in the art will readily appreciate that variants of these promoters can be used, such as promoters at least 95%, 96%, 97%, 98%, 99% identical to one of SEQ ID Nos: 1-3, provided the promoter functions, such that a heterologous nucleic acid operably linked to the promoter is expressed when transferred into a in a host cell that expresses glucagon. In additional aspects, the promoter can include at most 15, 14, 13, 12, 11, 10, 9, 8, 7, 6, 5, 4, 3, 2, 1 nucleic acid substitutions in SEQ ID NO: 1, provided the promoter functions, such that a heterologous nucleic acid operably linked to the promoter can be expressed when transferred into a in a host cell. In more aspects, the promoter can include at most 15, 14, 13, 12, 11, 10, 9, 8, 7, 6, 5, 4, 3, 2, 1 nucleic acid substitutions in SEQ ID NO: 2, provided the promoter functions, such that a heterologous nucleic acid operably linked to the promoter can be expressed when transferred into a in a host cell. In yet other aspects, the promoter can include at most 15, 14, 13, 12, 11, 10, 9, 8, 7, 6, 5, 4, 3, 2, 1 nucleic acid substitutions in SEQ ID NO: 3, provided the promoter functions, such that a heterologous nucleic acid operably linked to the promoter can be expressed when transferred into a in a host cell. Additional nucleotides can be added, provided the promoter functions, such that a heterologous nucleic acid operably linked to the promoter is expressed when transferred into a in a host cell. The promoter can include the nucleic acid sequence set forth as SEQ ID NO: 1 or 2, or 10, 20, 30, 40, 50, 60, 70, 80, 90, 100, 200, 300, 400, 500, 600, 700, or 800 nucleotides of SEQ ID NO: 1 and 2, provided the promoter functions to provide transcription of a nucleic acid encoding a heterologous protein, such as Pdx1 and/or MafA. The promoter can include the nucleic acid sequence set forth as SEQ ID NO: 3, or 10, 20, 30, 40, 50, 60, 70, 80, 90, 100, 200, 300, 400, 500, or 600 nucleotides of SEQ ID NO: 3, provided the promoter functions to provide transcription of a nucleic acid encoding a heterologous protein, such as Pdx1 and/or MafA.

Thus, in specific examples, the heterologous nucleic acid encodes Pdx1 and/or MafA. In additional examples, the heterologous nucleic acid encodes Pdx1 and/or MafA and does not encode Ngn3. In some aspects, the promoter functions such that both MafA and/or Pdx1 transcripts are produced. In specific non-limiting examples, the promoter is operably linked to a nucleic acid encoding MafA and a nucleic acid encoding Pdx1, but is not operably linked to a nucleic acid encoding Ngn3, and a vector including the promoter and the heterologous nucleic acid, such as rAAV vector, does not include a nucleic acid encoding Ngn3.

In some aspects, the vectors encode a MafA amino acid sequence including the amino acid sequence set forth as:

MAAELAMGAE LPSSPLAIEY VNDEDLMKFE VKKEPPEAER FCHRLPPGSL SSTPLSTPCS
SVPSSPSFCA PSPGTGGGGG AGGGGGSSQA GGAPGPPSGG PGAVGGTSGK
PALEDLYWMSGYQHHLNPEA LNLTPEDAVE ALIGSGHHGA HHGAHHPAAA AAYEAFRGPG
FAGGGGADDMGAGHHHGAHH AAHHHHAAHH HHHHHHHHGG AGHGGGAGHH VRLEERESDD
QLVSMSVRELNRQLRGFSKE EVIRLKOKRR ILKNRGYAQS CRFKRVQQRH ILESEKCQLQ
SQVEQLKLEV GRLAKERDLY KEKYEKLAGR GGPGSAGGAG FPREPSPPQA GPGGAKGTAD FFL

(human MafA, SEQ ID NO: 4, GENBANK® Accession No. NP_963883.2, May 10, 2014, incorporated herein by reference), or

MAAELAMGAE LPSSPLAIEY VNDEDLMKFE VKKEPPEAER FCHRLPPGSL SSTPLSTPCS
SVPSSPSFCA PSPGTGGGAG GGGSAAQAGG APGPPSGGPG TVGGASGKAV
LEDLYWMSGYQHHLNPEALN LTPEDAVEAL IGSGHHGAHH GAHHPAAAAA YEAFRGQSFA
GGGGADDMGAGHHHGAHHTA HHHHSAHHHH HHHHHHGGSG HHGGGAGHGG GGAGHHVRLE
ERFSDDQLVSMSVRELNRQL RGESKEEVIR LKQKRRTLKN RGYAQSCREK RVQQRHILES
EKCQLQSQVEQLKLEVGRLA KERDLYKEKY EKLAGRGGPG GAGGAGEPRE PSPAQAGPGA
AKGAPDFFL

(mouse MafA, SEQ ID NO: 5, GENBANK® Accession No. NP_919331, Apr. 26, 2014, incorporated herein by reference

MafA is a beta cell specific and glucose regulated transcription factor for insulin gene expression. The vector, such as an rAAV vector, can encode a MafA protein that has an amino acid sequence at least 95%, 96%, 97%, 98%, 99% or 100% identical to SEQ ID NO: 4 or SEQ ID NO: 5, wherein the protein functions as a transcription factor. The vector can encode a MafA protein that includes at most 10, 9, 8, 7, 6, 5, 4, 3, 2, or 1 conservative amino acid substitutions in SEQ ID NO: 4 or SEQ ID NO: 5, wherein the protein functions as a transcription factor. The vector can be a AAV vector.

In some aspects, the vectors encode a human Pdx1 amino acid sequence including the amino acid sequence set forth as:

MNGEEQYYAA TOLYKDPCAF QRGPAPEFSA SPPACLYMGR QPPPPPPHPF
PGALGALEQGSPPDISPYEV PPLADDPAVA HLHHHLPAQL ALPHPPAGPF PEGAEPGVLE
EPNRVQLPFPWMKSTKAHAW KGQWAGGAYA AEPEENKRTR TAYTRAQLLE LEKEFLENKY
ISRPRRVELAVMLNLTERHI KIWFQNRRMK WKKEEDKKRG GGTAVGGGGV AEPEQDCAVT
SGEELLALPPPPPPGGAVPP AAPVAAREGR LPPGLSASPQ PSSVAPRRPQ EPR

(human Pdx1, SEQ ID NO: 6, GENBANK Accession No. NP_000200.1, Mar. 15, 2015, incorporated herein by reference), or

MNSEEQYYAATQLYKDPCAFQRGPVPEFSANPPACLYMGRQPPPPPPPQ
FTSSLGSLEQGSPPDISPYEVPPLASDDPAGAHLHHHLPAQLGLAHPPP
GPFPNGTEPGGLEEPNRVQLPFPWMKSTKAHAWKGQWAGGAYTAEPEEN
KRTRTAYTRAQLLELEKEFLENKYISRPRRVELAVMLNLTERHIKIWFQ
NRRMKWKKEEDKKRSSGTPSGGGGGEEPEQDCAVTSGEELLAVPPLPPP
GGAVPPGVPAAVREGLLPSGLSVSPQPSSIAPLRPQEPR

(mouse Pdx1, SEQ ID NO: 7, GENBANK Accession No: NM_008814.3, Feb. 15, 2015, incorporated herein by reference).

Pdx1 is a transcriptional activator of several genes, including insulin, somatostatin, glucokinase, islet amyloid polypeptide, and glucose transportertype 2.

The vector, such as an rAAV vector, can encode a Pdx1 protein that has an amino acid sequence at least 95%, 96%, 97%, 98%, 99% or 100% identical to SEQ ID NO: 6 or SEQ ID NO: 7, wherein the protein functions as a transcription factor. The vector can encode a MafA protein that includes at most 10, 9, 8, 7, 6, 5, 4, 3, 2, or 1 conservative amino acid substitutions in SEQ ID NO: 6 or SEQ ID NO: 7, wherein the protein functions as a transcription factor. The vector can be a rAAV2, rAAV6, rAAV8 or rAAV9 vector.

In some aspects, the nucleic acid sequences encoding MafA and Pdx1 are separated by a connector. In specific non-limiting examples, the connector is 2A. The nucleic acid sequence of the 2A connector is shown below:

(SEQ ID NO: 8)
CGCGCCAAGCGCGGCTCCGGCGCCACCAACTTCTCCCTGCTGAAGCAG

Other exemplary connectors are:

(SEQ ID NO: 9)
CGCGCCAAGCGCGGCTCCGGCCAGTGCACCAACTACGCCCTGCTGAAGCT
GGCCGGCGACGTGGAGTCCAACCCCGGCCCC;
and
(SEQ ID NO: 10)
CGCGCCAAGCGCGGCTCCGGCGAGGGCAGAGGAAGTCTTCTAACATGCGG
TGACGTGGAGGAGAATCCCGGCCCT.

Suitable connectors also include a nucleic acid sequence with at most 1, 2, 3, 4, or 5 substitutions in one of SEQ ID NO: 8-10. Suitable connectors are, for example, 40 to 90 nucleotides in length, such as 45 to 85 nucleotides in length, such as 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 nucleotides in length.

The vectors of use in the method disclosed herein encode MafA and Pdx1, but do not encode Ngn3, for example, the Ngn3 protein of GENBANK® Accession No: NM_009719 (mouse), Feb. 15, 2015 and GENBANK® Accession No: NP_033849.3 (Human), Feb. 15, 2015. An exemplary Ngn3 protein is shown below:

(SEQ ID NO: 11)
MAPHPLDALT IQVSPETQQP FPGASDHEVL SSNSTPPSPT LIPRDCSEAE VGDCRGTSRK
LRARRGGRNR PKSELALSKQ RRSRRKKAND RERNRMHNLN SALDALRGVL
PTFPDDAKLTKIETLRFAHN YIWALTQTLR IADHSFYGPE PPVPCGELGS PGGGSNGDWG
SIYSPVSQAGNLSPTASLEE FPGLQVPSSP SYLLPGALVF SDFL

Thus, in some aspects, the vector, such as an rAAV vector, does not encode protein that has an amino acid sequence at least 95%, 96%, 97%, 98%, 99% or 100% identical to SEQ ID NO: 11, wherein the protein functions as a transcription factor. In further aspects, the vector does not encode a protein that includes at most 10, 9, 8, 7, 6, 5, 4, 3, 2, or 1 conservative amino acid substitutions in SEQ ID NO: 11, wherein the protein functions as a transcription factor.

U.S. Published Patent Application No. 2011/0280842, incorporated by reference herein, provides Pdx1, MafA and Ngn3 amino acid and nucleic acid sequence. In some aspects, a rAAV vector includes a nucleic acid encoding Pdx1 and MafA, but does not include a nucleic acid encoding Ngn3.

In some aspects, a vector of use includes a gene encoding a selectable marker, which includes, but are not limited to, a protein whose expression can be readily detected such as a fluorescent or luminescent protein or an enzyme that acts on a substrate to produce a colored, fluorescent, or luminescent substance (“detectable markers”). There are other genes of use, such as genes that encode drug resistance of provide a function that can be used to purify cells. Selectable markers include neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also selectable makers.

### Pharmaceutical Compositions, Methods, and Administration to the Pancreatic Duct

Methods are disclosed for treating T2DM in a subject. The subject can be any mammalian subject, including human and veterinary subjects. The subject can be an adult. The method can include selecting a subject of interest, such as a subject with T2DM.

The subject can also be administered metformin. The disclosed methods can also include having the subject make lifestyle modifications, such as increased physical activity, low fat diet, low sugar diet, and smoking cessation. In some aspects, the subject can be administered an effective dose of one or more lipid lowering compounds (such as statins or fibrates). The subject can be administered metformin. The subject can be obese, with a body mass index (BMI) of greater than or equal to 30. The subject ca be overweight with a body mass index of greater than or equal to 25 but less than 30. These subjects can be selected for treatment.

In some examples, a subject with T2DM may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. Additional information can be found in Standards of Medical Care in Diabetes—2010 (American Diabetes Association, Diabetes Care 33: S11-61, 2010, incorporated herein by reference).

The disclosed pharmaceutical compositions including can be delivered to humans or other animals by any means, including orally, intravenously, intramuscularly, intraperitoneally, intranasally, intradermally, intrathecally, subcutaneously, via inhalation or via suppository. In one non-limiting example, the composition is administered into the pancreatic duct of a subject in vivo.

These methods include administering to the subject a vector, such as an adenovirus vector or an AAV vector, inducing an inulin promoter operably linked to a nucleic acid molecule encoding heterologous Pdx1 and MafA. In some aspects, the vector does not include a nucleic acid encoding Neurogenin 3 (Ngn3). The subject is not administered any other nucleic acid encoding Ngn3.

Appropriate doses depend on the subject being treated (e.g., human, non-human primate, or other mammal), age and general condition of the subject to be treated, the severity of the condition being treated, the agent selected, and the mode of administration, among other factors. An appropriate effective amount can be readily determined by one of skill in the art. Thus, a “therapeutically effective amount” will fall in a relatively broad range that can be determined through clinical trials. The method can include measuring an outcome, such as insulin production, improvement in a fasting plasma glucose tolerance test, A1C level, oral glucose tolerance test, or symptoms of the subject. In some examples, administering the therapeutically effective amount of the inhibitor reduces plasma glucose levels or reduces glucose intolerance in the subject.

The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, PA, 21^{st} Edition (2005). For instance, parenteral formulations usually comprise injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate. Excipients that can be included are, for instance, other proteins, such as human serum albumin or plasma preparations.

For in vivo delivery, a vector, such as an adenovirus or an AAV vector can be formulated into a pharmaceutical composition and will generally be administered locally or systemically. In some aspects, the vector is administered directly to the pancreas. In other aspects, intraductally into a pancreatic duct of the subject. In other aspects, the subject has T2DM.

In some aspects, methods are provided for improving hyperglucagonemia, insulin sensitivity, and/or glucose homeostasis, respectively. These parameters can be improved as compared to a control, such as a standard value. In further aspects, the method improves hyperglucagonemia, insulin sensitivity, and/or glucose homeostasis in the subject as compared to a control value, wherein the control value is hyperglucagonemia, insulin sensitivity, and/or glucose homeostasis, respectively, in the subject prior to treatment with the vector.

In more aspects, the methods include administering to the subject a vector encoding Pdx1 and MafA, wherein the vector does not encode Ngn3 and wherein the subject is not administered any other nucleic acid encoding Ngn3. In yet other aspects, the vector is administered intraductally into a pancreatic duct of the subject.

The subject can be any mammalian subject, including human and veterinary subjects. The subject can be a child or an adult. The method can include selecting a subject of interest, such as a subject with T2DM. The subject can have pre-diabetes, or be at risk for developing T2DM. The subject can be overweight. The subject can be post-menopausal. The subject can have risk factor for T2DM.

In some aspects, the subject can also be administered metformin. In further aspects, the method can include measuring hyperglucagonemia, insulin sensitivity, and/or glucose homeostasis in the subject.

In some examples, a subject with diabetes may be clinically diagnosed by a fasting plasma glucose (FPG) concentration of greater than or equal to 7.0 millimole per liter (mmol/L) (126 milligram per deciliter (mg/dL)), or a plasma glucose concentration of greater than or equal to 11.1 mmol/L (200 mg/dL) at about two hours after an oral glucose tolerance test (OGTT) with a 75 gram (g) load, or in a patient with classic symptoms of hyperglycemia or hyperglycemic crisis, a random plasma glucose concentration of greater than or equal to 11.1 mmol/L (200 mg/dL), or HbA1c levels of greater than or equal to 6.5%. In other examples, a subject with pre-diabetes may be diagnosed by impaired glucose tolerance (IGT). An OGTT two-hour plasma glucose of greater than or equal to 140 mg/dL and less than 200 mg/dL (7.8-11.0 mM), or a fasting plasma glucose (FPG) concentration of greater than or equal to 100 mg/dL and less than 125 mg/dl (5.6-6.9 mmol/L), or HbA1c levels of greater than or equal to 5.7% and less than 6.4% (5.7-6.4%) is considered to be IGT, and indicates that a subject has pre-diabetes. Additional information can be found in Standards of Medical Care in Diabetes—2010 (American Diabetes Association, Diabetes Care 33: S11-61, 2010, incorporated herein by reference).

Appropriate doses depend on the subject being treated (e.g., human or nonhuman primate or other mammal), age and general condition of the subject to be treated, the severity of the condition being treated, the mode of administration of the AAV vector/virion, among other factors. An appropriate effective amount can be readily determined by one of skill in the art. Thus, a “therapeutically effective amount” will fall in a relatively broad range that can be determined through clinical trials. The method can include measuring an outcome, such as insulin production, improvement in a fasting plasma glucose tolerance test, or pancreatic beta cell number. The method can include administering other therapeutic agents, such as insulin. The method can also include having the subject make lifestyle modifications.

For example, for in vivo injection, i.e., injection directly to the subject, a therapeutically effective dose will be on the order of from about 10⁵ to 10¹⁶ of the AAV virions, such as 10⁸ to 10¹⁴ AAV virions. The dose, of course, depends on the efficiency of transduction, promoter strength, the stability of the message and the protein encoded thereby, and clinical factors. Effective dosages can be readily established by one of ordinary skill in the art through routine trials establishing dose response curves.

Dosage treatment may be a single dose schedule or a multiple dose schedule to ultimately deliver the amount specified above. Moreover, the subject may be administered as many doses as appropriate. Thus, the subject may be given, e.g., 10⁵ to 10¹⁶ AAV virions in a single dose, or two, four, five, six or more doses that collectively result in delivery of, e.g., 10⁵ to 10¹⁶ AAV virions. One of skill in the art can readily determine an appropriate number of doses to administer.

In some aspects, the AAV is administered at a dose of about 1×10¹¹ to about 1×10¹⁴ viral particles (vp)/kg. In some examples, the AAV is administered at a dose of about 1×10¹² to about 8×10¹³ vp/kg. In other examples, the AAV is administered at a dose of about 1×10¹³ to about 6×10¹³ vp/kg. In specific non-limiting examples, the AAV is administered at a dose of at least about 1×10¹¹, at least about 5×10¹¹, at least about 1×10¹², at least about 5×10¹², at least about 1×10¹³, at least about 5×10¹³, or at least about 1×10¹⁴ vp/kg. In other non-limiting examples, the AAV is administered at a dose of no more than about 5×10¹¹, no more than about 1×10¹², no more than about 5×10¹², no more than about 1×10¹³, no more than about 5×10¹³, or no more than about 1×10¹⁴ vp/kg. In one non-limiting example, the AAV is administered at a dose of about 1×10¹² vp/kg. The AAV can be administered in a single dose, or in multiple doses (such as 2, 3, 4, 5, 6, 7, 8, 9 or 10 doses) as needed for the desired therapeutic results, such as treatment of T2DM and/or improving improves hyperglucagonemia, insulin sensitivity, and/or glucose homeostasis in the subject.

Pharmaceutical compositions include sufficient genetic material to produce a therapeutically effective amount of MafA and Pdx1. In some aspects, AAV virions will be present in the subject compositions in an amount sufficient to provide a therapeutic effect, such as the production of pancreatic beta cells and/or the treatment of diabetes when given in one or more doses.

AAV virions can be provided as lyophilized preparations and diluted in a stabilizing composition for immediate or future use. Alternatively, the AAV virions can be provided immediately after production and stored for future use.

The pharmaceutical compositions can contain the vector, such as the rAAV vector, and/or virions, and a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

In some aspects, the excipients confer a protective effect on the AAV virion such that loss of AAV virions, as well as transduceability resulting from formulation procedures, packaging, storage, transport, and the like, is minimized. These excipient compositions are therefore considered “virion-stabilizing” in the sense that they provide higher AAV virion titers and higher transduceability levels than their non-protected counterparts, as measured using standard assays, see, for example, Published U.S. Application No. 2012/0219528, incorporated herein by reference. These Compositions therefore demonstrate “enhanced transduceability levels” as compared to compositions lacking the particular excipients described herein, and are therefore more stable than their non-protected counterparts.

Exemplary excipients that can used to protect the AAV virion from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, preferably, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients present in any of the disclosed compositions varies and is readily determined by one of skill in the art. For example, a protein excipient, such as BSA, if present, will can be present at a concentration of between 1.0 weight (wt.) % to about 20 wt. %, preferably 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. If a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous virion-stabilizing formulation comprises a carbohydrate, such as sorbitol, at a concentration of between 0.1 wt. % to about 10 wt. %, such as between about 1 wt. % to about 5 wt. %, and a detergent, such as a sorbitan ester (TWEEN®) at a concentration of between about 0.05 wt. % and about 5 wt. %, such as between about 0.1 wt. % and about 1 wt. %. Virions are generally present in the composition in an amount sufficient to provide a therapeutic effect when given in one or more doses, as defined above.

The pharmaceutical compositions can include a contrast dye is administered in addition to the viral vector, such an adenoviral vector, including an insulin promoter operably linked to a nucleic acid molecule encoding Pdx1 and MafA. The contrast dye can be a low-osmolar low-viscosity non-ionic dye, a low-viscosity high-osmolar dye, or a dissociable high-viscosity dye. In specific non-limiting examples the dye is Iopromid, Ioglicinate, or Ioxaglinate. Thus, provided herein is a pharmaceutical composition including a) an adeno-associated virus vector, such as rAAV8, comprising a promoter operably linked to a nucleic acids encoding Pdx1 and a nucleic acid encoding MafA, wherein the vector does not encode Ngn3; b) a buffer; and c) a contrast dye for endoscopic retrograde cholangiopancreatography. In some aspects, the pharmaceutical composition does not include a nucleic acid encoding Ngn3. Any of the AAV vectors disclosed herein can be included in this composition. The AAV vector can be encapsulated in a virion. The composition can be formulated for administration to the pancreatic duct.

The disclosed pharmaceutical compositions including a therapeutically effective amount of a viral vector, such an adenoviral vector, including an insulin promoter operably linked to a nucleic acid molecule encoding Pdx1 and MafA, or a virion, can be delivered to humans or other animals by any means, including orally, intravenously, intramuscularly, intraperitoneally, intranasally, intradermally, intrathecally, subcutaneously, via inhalation or via suppository. In one non-limiting example, the composition is administered into the pancreatic duct of a subject in vivo.

One exemplary method for intraductal administration is Endoscopic Retrograde Cholangiopancreatography (ERCP). ERCP is an endoscopic technique that involves the placement of a side-viewing instrument (generally either an endoscope or duodenoscope) within the descending duodenum. The procedure eliminates the need for invasive surgical procedures for administration to the pancreatic duct.

In an ERCP procedure, the patient will generally lie on their side on an examining table. The patient will then be given medication to help numb the back of the patient's throat, and a sedative to help the patient relax during the examination. The patient then swallows the endoscope. The thin, flexible endoscope is passed carefully through the alimentary canal of the patient. The physician guides the endoscope through the patient's esophagus, stomach, and the first part of the small intestine known as the duodenum. Because of the endoscope's relatively small diameter, most patients can tolerate the unusualness of having the endoscope advanced through the opening of their mouth.

The physician stops the advancement of the endoscope when the endoscope reaches the junction where the ducts of the biliary tree and pancreas open into the duodenum. This location is called the papilla of Vater, or also commonly referred to as the ampulla of Vater. The papilla of Vater is a small mound of tissue looking and acting similarly to a nipple. The papilla of Vater emits a substance known as bile into the small intestine, as well as pancreatic secretions that contain digestive enzymes. Bile is a combination of chemicals made in the liver and is necessary in the act of digestion. Bile is stored and concentrated in the gallbladder between meals. When digestive indicators stimulate the gallbladder, however, the gallbladder squeezes the bile through the common bile duct and subsequently through the papilla of Vater. The pancreas secretes enzymes in response to a meal, and the enzymes help digest carbohydrates, fats, and proteins.

The patient will be instructed (or manually maneuvered) to lie flat on their stomach once the endoscope reaches the papilla of Vater. For visualization or treatment within the biliary tree, the distal end of the endoscope is positioned proximate the papilla of Vater. A catheter is then advanced through the endoscope until the distal tip of the catheter emerges from the opening at the endoscope's distal end. The distal end of the catheter is guided through the endoscope's orifice to the papilla of Vater (located between the sphincter of Oddi) leading to the common bile duct and the pancreatic duct. In the case of pancreas-specific delivery of reagents, the pancreatic duct proper can be entered.

ERCP catheters can be constructed from Teflon, polyurethane and polyaminde. ERCP catheters also can also be constructed from resin comprised of nylon and PEBA (see U.S. Pat. No. 5,843,028), and can be construed for use by a single operator (see U.S. Pat. No. 7,179,252). At times, a spring wire guide may be placed in the lumen of the catheter to assist in cannulation of the ducts. A stylet, used to stiffen the catheter, must first be removed prior to spring wire guide insertion.

A dual or multi-lumen ERCP catheter in which one lumen could be utilized to accommodate the spring wire guide or diagnostic or therapeutic device, and in which a second lumen could be utilized for contrast media and/or dye infusion and or for administration of a pharmaceutical composition including a viral vector, such an adenoviral vector, encoding Pdx1 and MafA. In some aspects, a contrast dye is administered in addition to the pharmaceutical composition including a viral vector, such an adenoviral vector, encoding Pdx1 and MafA. The contrast dye can be a low-osmolar low-viscosity non-ionic dye, a low-viscosity high-osmolar dye, or a dissociable high-viscosity dye. In specific non-limiting examples the dye is Iopromid, Ioglicinate, or loxaglinate. Endoscopes have been designed for the delivery of more than one liquid solution, such as a first liquid composition including a viral vector, such an adenoviral vector, encoding Pdx1 and MafA, and a second liquid composition including dye, see U.S. Pat. No. 7,597,662, which is incorporated herein by reference. Thus, the pharmaceutical composition including a viral vector, such an adenoviral vector, encoding Pdx1 and MafA and the dye can be delivered in the same or separate liquid compositions. Methods and devices for using biliary catheters for accessing the biliary tree for ERCP procedures are disclosed in U.S. Pat. Nos. 5,843,028, 5,397,302 5,320,602, which are incorporated by reference herein.

In additional examples, the vector is administered using a viral infusion technique into a pancreatic duct. Suitable methods are disclosed, for example, in Guo et al. Laboratory Invest. 93:1241-1253, 2013, incorporated by reference herein.

### Examples

Six-week C57BL/6J male mice were placed on a high-fat diet (HFD) or regular diet (RD). After 16 weeks of HFD, intraperitoneal glucose tolerance testing (IPGTT) confirmed hyperglycemia in 100% of the HFD mice. HFD mice received AAV-CMV-PM-GFP (treatment with CMV promoter), AAV-RIP-PM-GFP (treatment with Rat Insulin Promoter) or AAV-CMV-GFP (virus control) or remained unoperated. Diet remained unchanged after the surgery.

At 2 weeks post-infusion, HFD groups were not significantly different in body weight, and all of them were significantly heavier than the RD group (FIG. 1A). The (HFD+AAV-RIP-PM-GFP) had improved glucose tolerance compared to all other HFD groups, including the AAV-CMV-PM-GFP. There was no difference in glucose tolerance between HFD+AAV-RIP-PM-GFP mice and regular diet mice (i.e. the glucose tolerance was completely normalized, FIG. 1B,C). Glucose-stimulated insulin secretion (GSIS) obtained during the IPGTT revealed a significant increase in the insulin secretion in all HFD groups compared to regular diet mice (FIG. 1D). A glucose load significantly decreased glucagon levels at 30 minutes compared to fasting glucagon levels in RD mice and (HFD+AAV-RIP-PM-GFP), but not in the other HFD groups (FIG. 1E), suggesting amelioration of the hyperglucagonemia, likely due to improved insulin secretion.

The HOMA-IR, a marker of insulin resistance, calculated using the fasting glucose and fasting insulin obtained during the IPGTT (FIG. 1F), showed an expected decreased insulin sensitivity in (HFD alone) and (HFD+AAV-CMV-GFP) compared to the regular diet group. However, the (HFD+AAV-CMV-PM-GFP) and (HFD+AAV-RIP-PM-GFP) had similar insulin sensitivity, they were both not different from either regular diet and HFD mice, indicating partial improvement in insulin sensitivity in the PM-treated mice. Overall, these results show that the insulin promoter induces a more rapid improvement in glucose homeostasis than the CMV promoter.

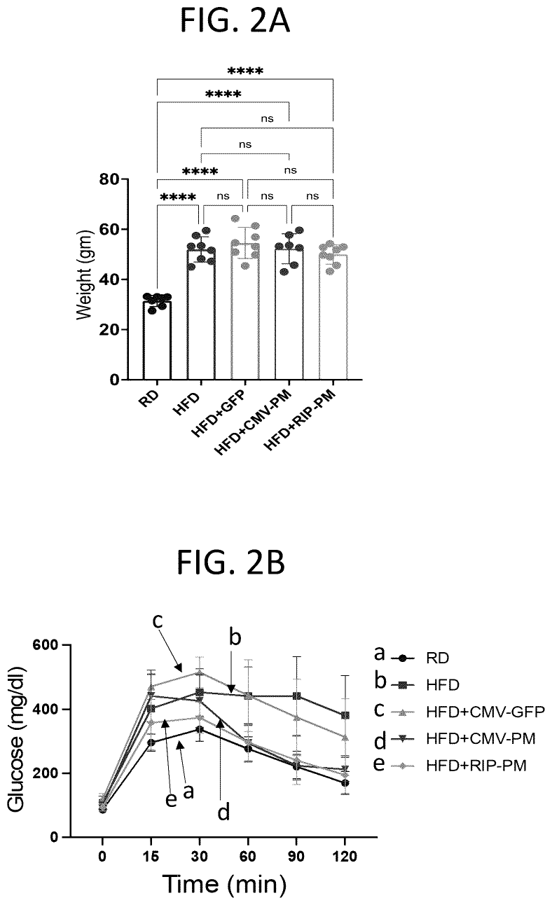

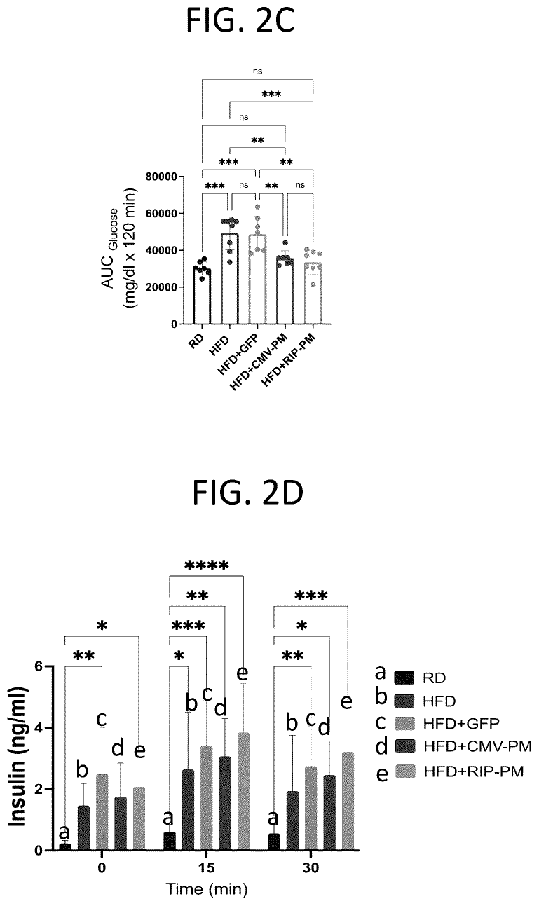

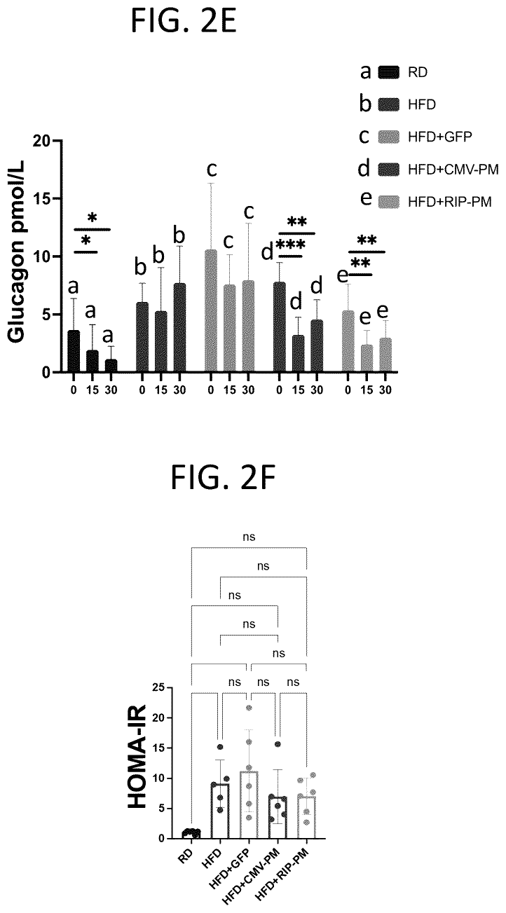

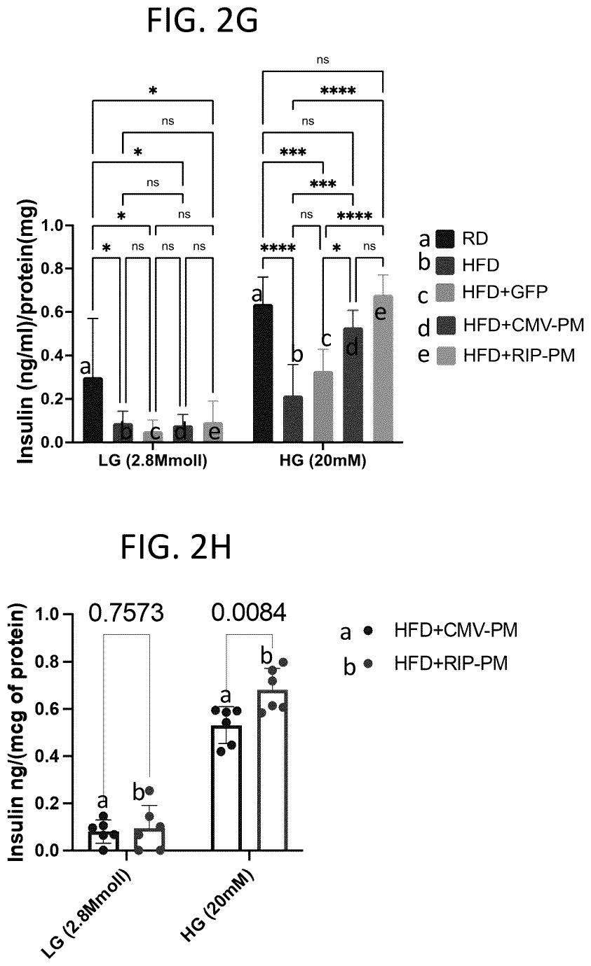

At 4 weeks, HFD groups were not significantly different in body weight, and all of them were significantly heavier than the RD group (FIG. 2A). IPGTT at 4 weeks post-surgery showed a significant improvement in the glucose tolerance of (HFD+AAV-CMV-PM-GFP) and (HFD+AAV-RIP-PM-GFP) compared to (HFD+AAV-GFP) and HFD alone. There was no difference between (HFD+AAV-CMV-PM-GFP), (HFD+AAV-RIP-PM-GFP) and RD mice (FIGS. 2A, 2B), suggesting complete rescue of the HFD-induced hyperglycemia. GSIS obtained during the IPGTT revealed a significant increase in insulin secretion in all HFD groups compared to regular diet mice (FIG. 2D). Also, a glucose load significantly decreased glucagon levels at 30 minutes compared to fasting glucagon levels in RD mice and (HFD+AAV-CMV-PM-GFP) and (HFD+AAV-CMV-PM-GFP), but not in the HFD control groups (FIG. 2E), suggested amelioration of the hyperglucagonemia, likely due to improved insulin secretion. The HOMA-IR calculated using the fasting glucose and fasting insulin obtained during the IPGTT (FIG. 2F) showed an expected decreased insulin sensitivity in (HFD alone) and (HFD+AAV-CMV-GFP) compared to the regular diet group. However, the (HFD+AAV-CMV-PM-GFP) and (HFD+AAV-RIP-PM-GFP) had similar insulin sensitivity, they were both not different from either regular diet or HFD mice, indicating partial improvement in insulin sensitivity in the 2 groups of PM-treated mice. Pancreatic islets isolated from the 5 groups for ex-vivo GSIS showed that islets isolated from (HFD+AAV-CMV-PM-GFP) and (HFD+AAV-RIP-PM-GFP) had insulin secretion similar to islets isolated from RD mice, and both groups had increased insulin secretion compared to islets isolated from control HFD groups (FIG. 2G). Furthermore, ex-vivo GSIS of islets isolated from (HFD+AAV-RIP-PM-GFP) had significantly higher insulin secretion than islets isolated from (HFD+AAV-RIP-PM-GFP). Both treatments completely normalized the blood glucose physiology, but the insulin promoter was much faster. The insulin promoter normalized blood glucose in 2 weeks, and blood glucose stayed normalized for at least 8 weeks. The CMV promoter required 4 weeks for blood glucose to normalize.

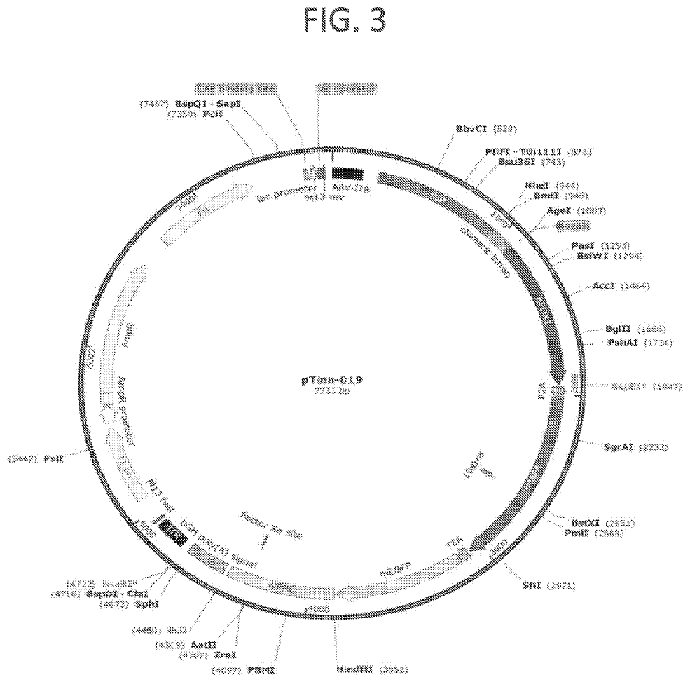

FIG. 3 shows a construct used for these studies.

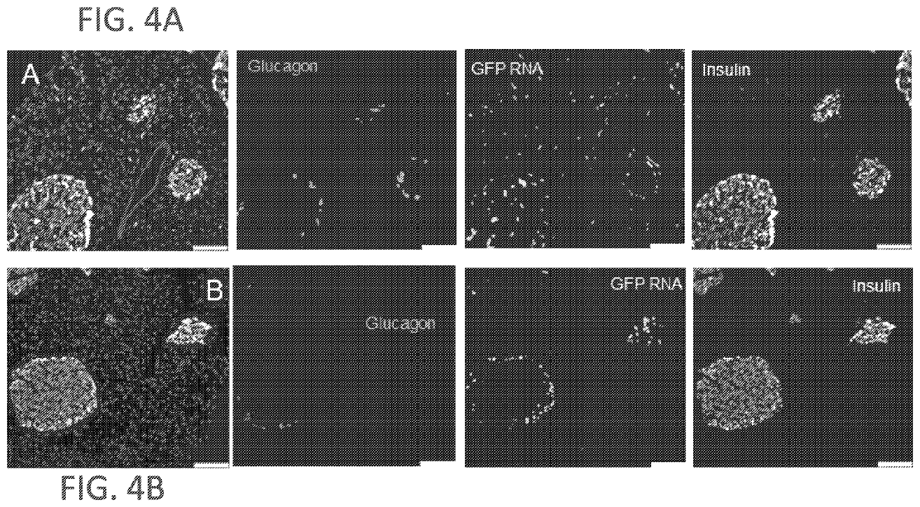

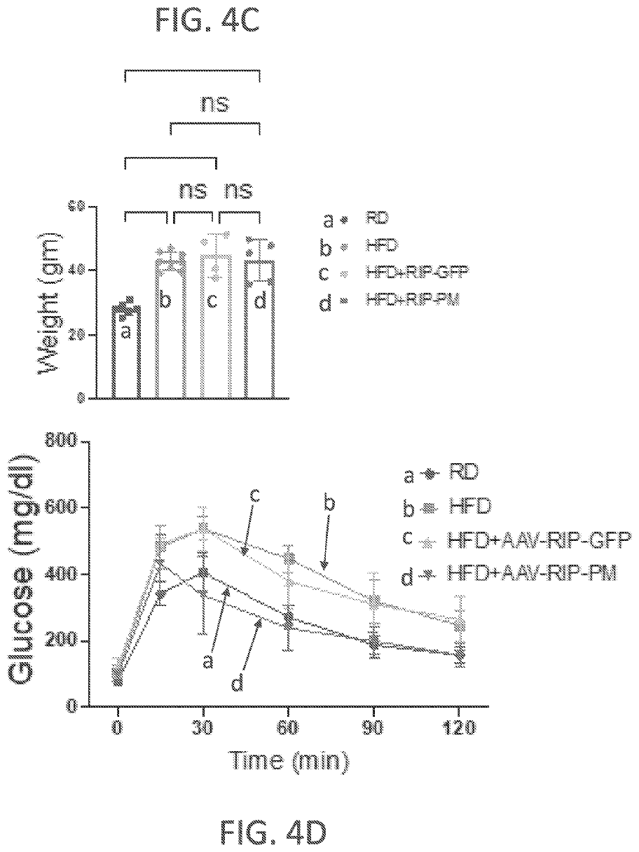

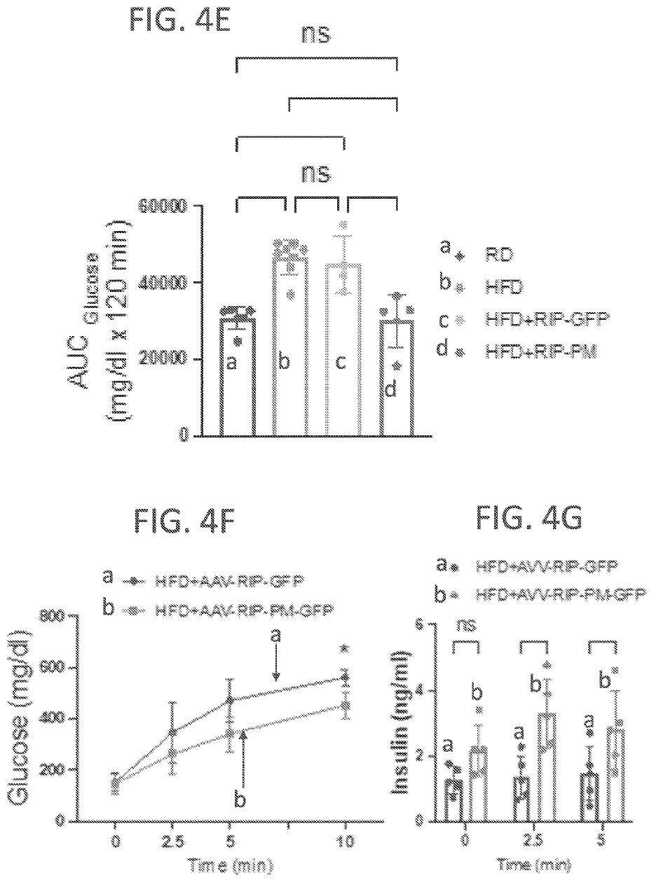

The RNASCOPE® was determined for the green fluorescent protein (GFP) RNA combined with immunostaining for insulin and glucagon in a pancreas infused with either AAV-CMV-PM-GFP (FIG. 4A) or AAV-RIP-PM-GFP (FIG. 4B). This illustrates how the insulin promoter confers specificity on the beta cells, as the GFP RNA distribution in FIG. 4B overlaps only with the insulin-positive cells. As shown in FIG. 4C, comparison between mouse weights in the 4 groups at 4 weeks shows that the virus does not affect weight gain. IPGTT at 4 weeks shows normalization of the glucose tolerance curve (FIG. 4D). FIG. 4E shows the area under the curve (AUC). Glucose excursion at very early time points during the in vivo GSIS is shown in FIG. 4F. The first phase insulin secretion is improved, which is important because the first phase insulin secretion is the most serious problem in Type 2 diabetes. FIG. 4G shows the insulin levels from the experiment in FIG. 4F, again showing the markedly improved first phase insulin secretion.

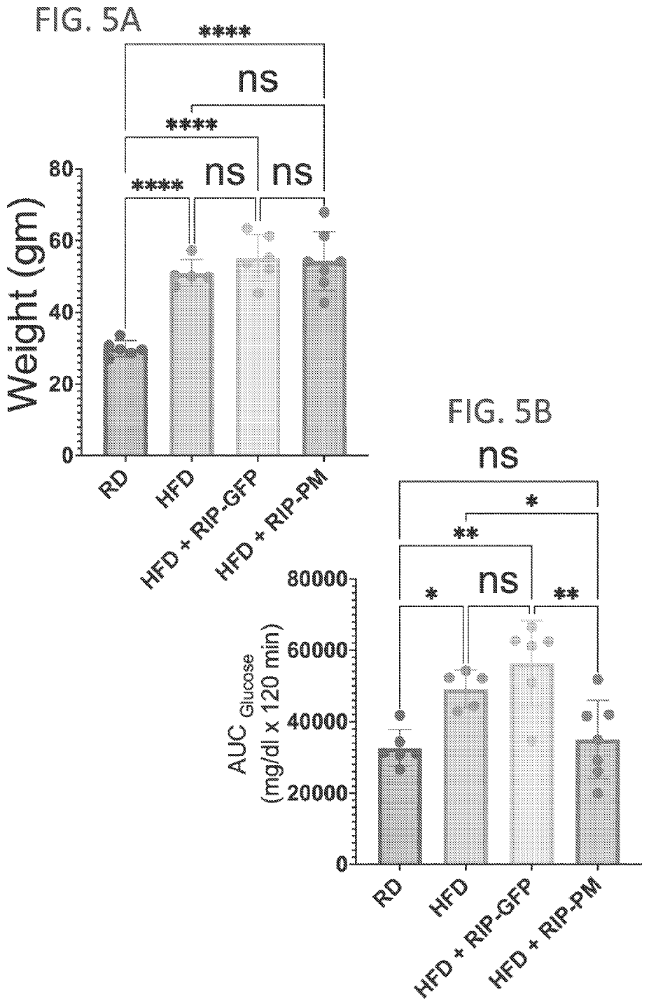

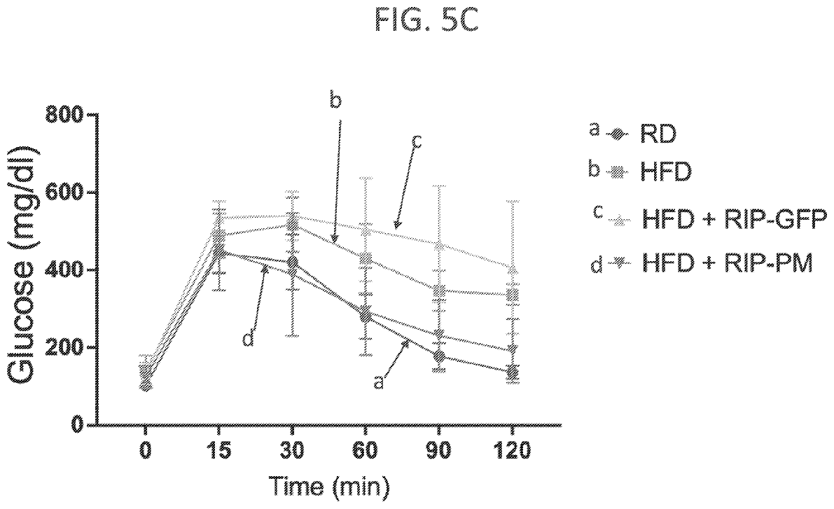

FIGS. 5A-5C provide additional data. At five months after virus infusion using the RIP promoter, the animals are still euglycemic. The body weight remains high (FIG. 5A) as they have been maintained on the high fat diet, but despite that the glucose tolerance remains equivalent to animals maintained on a regular chow diet. Area under the curve (AUC) shown in FIG. 5B and glucose tolerance testing curve in FIG. 5C. The CMV promoter effect only worked for 4-6 weeks after the infusion.

For the results shown in FIGS. 4A-4G and FIGS. 5A-5C, the following methods were used:

Mice: 7-8-week-old female CD1 mice were purchased from the Charles River Labs. The mice were maintained in an environment with a 12-hour light/dark cycle with the lights on from 7 AM to 7 PM. Intraductal pancreatic infusion of 150 μL of 1% AA was infused at 50 μL/min as described (23). All mice were maintained on elemental diet (Peptamen 1.0 CAL/55620A) mixed with mushed normal chow 1:1.

In vivo glucose homeostasis studies: IPGTT and GSIS: Overnight 16-hour-fasted mice were injected with 2 g/kg glucose (Sigma-Aldrich, St. Louis, MO). Blood glucose levels were detected at 0, 15, 30, 60, 90, and 120 minutes after injection using a glucometer (CONTOUR® NEXT EZ). During IPGTT, tail vein blood was collected at 0, 15 and 30 minutes or 0,2,5, and 10 minutes after glucose injection for measuring serum insulin concentration using an ELISA kit (ALPCO®, Salem, NH, USA).

RNA scope procedure: Paraffin-fixed tissues were heated in an oven at 60° C. for 1 hour, followed by deparaffinization at room temperature using xylene and 100% ethanol. After deparaffinization, RNASCOPE™ Hydrogen Peroxide was applied to each slide; H₂O₂-covered slides were left to incubate for 10 minutes at room temperature. Slides were then placed in a co-detection target retrieval solution for 15 minutes under boiling heat (99° C.-100° C.), after which they were washed with dH₂O. Primary antibodies for insulin and glucagon protein were then applied and slides incubated overnight at 4° C. On the second day, slides were placed in 10% neutral buffered formalin for 30 minutes at room temperature. After washing in phosphate buffered saline with TWEEN® 20 (PBST_, RNASCOPE™ Protease Plus were applied over the tissues and incubated in oven at 40° C. for 30 minutes. For RNASCOPE™, the GFP probe was applied (incubated in oven for 2 hours at 40° C.). Slides were then washed in RNAs RNASCOPE™ cope wash buffer for 2 minutes. A signal amplifier was then applied (incubated in oven for 30 minutes at 40° C.), followed by washing. This step was repeated 2 times. To develop HRP-C1 Signal, Opal Dye Fluorophore was applied (incubated in oven for 15 min at 40° C.), followed by washing. Lastly, an horse radish peroxidase (HRP)-blocker was applied (incubated in oven for 15 min at 40° C.), followed by washing. The GFP probe secondary antibody was then added to each slide (30 minutes at room temperature), followed by diamidino-2-phenylindole (DAPI) for 30 seconds, then Prolong Gold antifade mounting medium was applied for 30 minutes. Slides were stored at 2-8° C. in the dark.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method for treating type 2 diabetes mellites (T2DM) in a subject, comprising
administering to the subject a therapeutically effective amount of a vector comprising an insulin promoter operably linked to a nucleic acid molecule encoding heterologous Pancreas duodenal homeobox protein (Pdx) 1 and a nucleic acid molecule encoding Musculoaponeurotic fibrosarcoma oncogene homolog A (MafA);
wherein the vector is administered intraductally into a pancreatic duct of the subject,
thereby treating the T2DM in the subject.

2. The method of claim 1, wherein the vector does not encode Neurogenin 3 (Ngn3) and wherein the subject is not administered any other nucleic acid encoding Ngn3

3. The method of claim 1, wherein the vector is an adenovirus vector or an adeno-associated virus vector.

4. The method of claim 1, wherein the insulin promoter comprises the nucleic acid sequence of SEQ ID NO: 1, SEQ ID NO: 2 or SEQ ID NO: 3.

5. The method of claim 1, wherein the insulin promoter consists of the nucleotide sequence of SEQ ID NO: 1, SEQ ID NO: 2, or SEQ ID NO: 3.

6. The method of claim 1, wherein the nucleic acid molecule encoding Pdx1 and the nucleic acid molecule encoding MafA are linked with a connector.

7. The method of claim 6, wherein the connector comprises SEQ ID NO: 8.

8. The method of any claim 1, wherein the vector is administered using endoscopic retrograde cholangiopancreatography (ERCP).

9. The method of claim 1, wherein the subject is human.

10. The method of claim 1, wherein the subject is administered metformin.

11. The method of claim 1, wherein the method improves hyperglucagonemia, insulin sensitivity, and/or glucose homeostasis in the subject as compared to a control value, wherein the control value is hyperglucagonemia, insulin sensitivity, and/or glucose homeostasis, respectively, in the subject prior to treatment with the vector.

12. A composition comprising:
a) an adeno-associated virus vector comprising an insulin promoter operably linked to a nucleic acid molecule encoding Pdx1 and a nucleic acid molecule encoding MafA,
b) a buffer; and
c) a contrast dye for endoscopic retrograde cholangiopancreatography.

13. The composition of claim 12, wherein the composition does not comprise a nucleic acid encoding Ngn3 or Ngn3 polypeptide.

14. The composition of claim 12, wherein the insulin promoter comprises the nucleic acid sequence set forth as SEQ ID NO: 1, SEQ ID NO: 2, or SEQ ID NO: 3.

15. The composition of claim 12, wherein the insulin promoter consists of the nucleic acid sequence set forth as SEQ ID NO: 1, SEQ ID NO: 2 or SEQ ID NO: 3.

16. The composition of claim 12, wherein the contrast dye is a low-osmolar low-viscosity non-ionic dye, a low-viscosity high-osmolar dye, or a dissociable high-viscosity dye.

17. The composition of claim 16, wherein the contrast dye is Iopromid, Ioglicinate, or Ioxaglinate.

18. The composition of claim 12, formulated for administration to the pancreatic duct.

19. The composition of claim 12, wherein the nucleic acid sequence encoding Pdx1 and the nucleic acid sequence encoding MafA are linked using a connector.

20. The composition of claim 19, wherein the connector comprises SEQ ID NO: 8.

21. The composition of claim 12, wherein the adeno-associated virus vector comprises a nucleic acid sequence encoding a label.

22. The composition of claim 12, formulated for administration by endoscopic retrograde cholangiopancreatography.

23. The composition of claim 12, for use in treating type 2 diabetes.

24-26. (canceled)


SEQUENCE LISTING (11 sequences)

SEQ ID NO: 1 - DNA, 251 bp, Homo sapiens
  source 1..251: mol_type = genomic DNA
tcctgaggaagaggtgctgacgaccaaggagatcttcccacagacccagcaccagggaaatggtccggaaattgcagcctcagcccccagccatctgccgacccccccaccccaggccctaatgggccaggcggcaggggttgagaggtaggggagatgggctctgagactataaagccagcgggggcccagcagccctcagccctccaggacaggctgcatcagaagaggccatcaagcaggtctgttcc

SEQ ID NO: 2 - DNA, 422 bp, Homo sapiens
  source 1..422: mol_type = genomic DNA
gagctctgtggggacaggggtctggggacagcagcgcaaagagccccgccctgcagcctccagctctcctggtctaatgtggaaagtggcccaggtgagggctttgctctcctggagacatttgcccccagctgtgagcagggacaggtctggccaccgggcccctggttaagactctaatgacccgctggtcctgaggaagaggtgctgacgaccaaggagatcttcccacagacccagcaccagggaaatggtccggaaattgcagcctcagcccccagccatctgccgacccccccaccccaggccctaatgggccaggcggcaggggttgacaggtaggggagatgggctctgagactataaagccagcgggggcccagcagccctcagccctccaggacaggctgcatcagaagctt

SEQ ID NO: 3 - DNA, 688 bp, Rattus norvegicus
  source 1..688: mol_type = genomic DNA
caagtggaggctgagaaaggttttgtagctgggtagagtatgtactaagagatggagacagctggctctgagctctgaagcaagcacctcttatggagagttgctgaccttcaggtgcaaatctaagatactacaggagaatacaccatggggcttcagcccagttgactcccgagtgggctatgggtttgtggaaggagagatagaagagaagggacctttcttcttgaattctgctttccttctacctctgagggtgagctggggtctcagctgaggtgaggacacagctatcagtgggaactgtgaaacaacagttcaagggacaaagttactaggtcccccaacaactgcagcctcctggggaatgatgtggaaaaatgctcagccaaggacaaagaaggcctcaccctctctgagacaatgtcccctgctgtgaactggttcatcaggccacccaggagcccctattaagactctaattaccctaaggctaagtagaggtgttgttgtccaatgagcactttctgcagacctagcaccaggcaagtgtttggaaactgcagcttcagcccctctggccatctgctgatccacccttaatgggacaaacagcaaagtccaggggtcagggggggggtgctttggactataaagctagtggggattcagtaacccccagccctaa

SEQ ID NO: 4 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 5 - protein, 359 aa, Mus musculus
MAAELAMGAELPSSPLAIEYVNDFDLMKFEVKKEPPEAERFCHRLPPGSLSSTPLSTPCSSVPSSPSFCAPSPGTGGGAGGGGSAAQAGGAPGPPSGGPGTVGGASGKAVLEDLYWMSGYQHHLNPEALNLTPEDAVEALIGSGHHGAHHGAHHPAAAAAYEAFRGQSFAGGGGADDMGAGHHHGAHHTAHHHHSAHHHHHHHHHHGGSGHHGGGAGHGGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGGAGGAGFPREPSPAQAGPGAAKGAPDFFL

SEQ ID NO: 6 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 7 - protein, 284 aa, Mus musculus
MNSEEQYYAATQLYKDPCAFQRGPVPEFSANPPACLYMGRQPPPPPPPQFTSSLGSLEQGSPPDISPYEVPPLASDDPAGAHLHHHLPAQLGLAHPPPGPFPNGTEPGGLEEPNRVQLPFPWMKSTKAHAWKGQWAGGAYTAEPEENKRTRTAYTRAQLLELEKEFLFNKYISRPRRVELAVMLNLTERHIKIWFQNRRMKWKKEEDKKRSSGTPSGGGGGEEPEQDCAVTSGEELLAVPPLPPPGGAVPPGVPAAVREGLLPSGLSVSPQPSSIAPLRPQEPR

SEQ ID NO: 8 - DNA, 48 bp, synthetic construct
  source 1..48: mol_type = other DNA
cgcgccaagcgcggctccggcgccaccaacttctccctgctgaagcag

SEQ ID NO: 9 - DNA, 81 bp, synthetic construct
  source 1..81: mol_type = other DNA
cgcgccaagcgcggctccggccagtgcaccaactacgccctgctgaagctggccggcgacgtggagtccaaccccggcccc

SEQ ID NO: 10 - DNA, 75 bp, synthetic construct
  source 1..75: mol_type = other DNA
cgcgccaagcgcggctccggcgagggcagaggaagtcttctaacatgcggtgacgtggaggagaatcccggccct

SEQ ID NO: 11 - protein, 214 aa, Homo sapiens
MAPHPLDALTIQVSPETQQPFPGASDHEVLSSNSTPPSPTLIPRDCSEAEVGDCRGTSRKLRARRGGRNRPKSELALSKQRRSRRKKANDRERNRMHNLNSALDALRGVLPTFPDDAKLTKIETLRFAHNYIWALTQTLRIADHSFYGPEPPVPCGELGSPGGGSNGDWGSIYSPVSQAGNLSPTASLEEFPGLQVPSSPSYLLPGALVFSDFL